SEQUENCE LISTING (44 sequences)

SEQ ID NO: 1 - DNA, 5413 bp, Artificial Sequence
  Barcoded AAV capsid plasmid (pZac-CASI-eGFP- barcode-PolyA)
  misc_feature (1)..(130): ITR sequence
  promoter (197)..(1252): CASI promoter sequence
  misc_feature (1268)..(1987): eGFP gene sequence
  misc_feature (2002)..(2009): AAV barcode 1
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgatcacgacaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 2 - DNA, 7911 bp, Artificial Sequence
  Plasmid for recombinant capsid libraries (pZac-ITR-Rep-rCap-PolyA-ITR)
  misc_feature (6)..(135): ITR sequence
  promoter (201)..(331): P5 promoter sequence
  misc_feature (332)..(2197): REP gene sequence
  misc_feature (2214)..(4424): recombinant CAP gene sequence
  misc_feature (4219)..(4905): Polyadenylation tail sequence
taaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaat

SEQ ID NO: 3 - DNA, 8310 bp, Artificial Sequence
  pSub201
  misc_feature (7)..(136): ITR sequence
  misc_feature (320)..(2185): REP gene sequence
  misc_feature (2202)..(4410): recombinant CAP gene sequence
  misc_feature (4547)..(4674): ITR sequence
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgtcaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccggggcccgccatggcaagccacaaggacgatgaagaaaagttttttcctcagagcggggttctcatctttgggaagcaaggctcagagaaaacaaatgtgaacattgaaaaggtcatgattacagacgaagaggaaatcggaacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 4 - DNA, 6867 bp, Artificial Sequence
  pZac-ITR-CASI-rCAP-PolyA-ITR
  misc_feature (1)..(130): ITR sequence
  promoter (197)..(1252): CASI promoter sequence
  misc_feature (1254)..(3464): recombinant CAP gene sequence
  misc_feature (3470)..(3856): Polyadenylation tail sequence
  misc_feature (3916)..(4056): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 5 - DNA, 130 bp, Artificial Sequence
  ITR sequence of barcoded AAV capsid plasmids (pZac-CASI-eGFP-barcode-PolyA), Plasmid for recombinant capsid libraries (pZac-ITR-Rep-rCap-PolyA-ITR) and pSub201
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, Artificial Sequence
  ITR sequence of barcoded AAV capsid plasmids (pZac-CASI-eGFP-barcode-PolyA), Plasmid for recombinant capsid libraries (pZac-ITR-Rep-rCap-PolyA-ITR) and pZac-ITR-CASI-rCAP-PolyA-ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaagg

SEQ ID NO: 7 - DNA, 128 bp, Artificial Sequence
  ITR sequence of pSub201
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 8 - DNA, 1056 bp, Artificial Sequence
  CASI promoter sequence of barcoded AAV capsid plasmids (pZac-CASI-eGFP-barcode-PolyA) and pZac-ITR-CASI-rCAP- PolyA-ITR
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 9 - DNA, 720 bp, Artificial Sequence
  eGFP gene sequence of Barcoded AAV capsid plasmids (pZac-CASI-eGFP-barcode-PolyA)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - DNA, 387 bp, Artificial Sequence
  Polyadenylation tail sequence of barcoded AAV capsid plasmid (pZac-CASI-eGFP-barcode-PolyA), pZac-ITR-CASI-rCAP- PolyA-ITR and plasmid for recombinant capsid libraries (pZac-ITR- Rep-rCap-PolyA-ITR)
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 11 - DNA, 131 bp, Artificial Sequence
  P5 promoter sequence of plasmid for recombinant capsid libraries (pZac-ITR-Rep-rCap-PolyA-ITR)
ggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 12 - DNA, 1866 bp, Artificial Sequence
  REP gene sequence of plasmid for recombinant capsid libraries (pZac-ITR-Rep-rCap-PolyA-ITR)
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataa

SEQ ID NO: 13 - DNA, 1866 bp, Artificial Sequence
  REP gene sequence of pSub201
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 2211 bp, Artificial Sequence
  Recombinant CAP gene sequence of plasmid for recombinant capsid libraries (pZac-ITR-Rep-rCap-PolyA-ITR)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 15 - DNA, 2209 bp, Artificial Sequence
  recombinant CAP gene sequence of pSub201
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgtcaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccggggcccgccatggcaagccacaaggacgatgaagaaaagttttttcctcagagcggggttctcatctttgggaagcaaggctcagagaaaacaaatgtgaacattgaaaaggtcatgattacagacgaagaggaaatcggaacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaa

SEQ ID NO: 16 - DNA, 2211 bp, Artificial Sequence
  recombinant CAP gene sequence of pZac-ITR-CASI- rCAP-PolyA-ITR
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 17 - DNA, 19 bp, Artificial Sequence
  Edit genome.fa reference GFP1 (with barcode sequence 1)
  misc_feature (12)..(19): Barcode sequence 1
taaatcgatcgatcacgac

SEQ ID NO: 18 - DNA, 19 bp, Artificial Sequence
  Edit genome.fa reference GFP2 (with barcode sequence 2)
  misc_feature (12)..(19): Barcode sequence 2
taaatcgatcgacagtggt

SEQ ID NO: 19 - DNA, 19 bp, Artificial Sequence
  Edit genome.fa reference GFP3 (with barcode sequence 3)
  misc_feature (12)..(19): Barcode sequence 3
taaatcgatcgcagatcca

SEQ ID NO: 20 - DNA, 19 bp, Artificial Sequence
  Edit genome.fa reference GFP4 (with barcode sequence 4)
  misc_feature (12)..(19): Barcode sequence 4
taaatcgatcgacaaacgg

SEQ ID NO: 21 - DNA, 19 bp, Artificial Sequence
  Edit genome.fa reference GFP5 (with barcode sequence 5)
  misc_feature (12)..(19): Barcode sequence 5
taaatcgatcgacccagca

SEQ ID NO: 22 - DNA, 19 bp, Artificial Sequence
  Edit genome.fa reference GFP6 (with barcode sequence 6)
  misc_feature (12)..(19): Barcode sequence 6
taaatcgatcgaacccctc

SEQ ID NO: 23 - DNA, 19 bp, Artificial Sequence
  Edit genome.fa reference GFP7 (with barcode sequence 7)
  misc_feature (12)..(19): Barcode sequence 7
taaatcgatcgcccaacct

SEQ ID NO: 24 - DNA, 19 bp, Artificial Sequence
  Edit genome.fa reference GFP8 (with barcode sequence 8)
  misc_feature (12)..(19): Barcode sequence 8
taaatcgatcgcaccacac

SEQ ID NO: 25 - DNA, 19 bp, Artificial Sequence
  Edit genome.fa reference GFP9 (with barcode sequence 9)
  misc_feature (12)..(19): Barcode sequence 9
taaatcgatcggaaaccca

SEQ ID NO: 26 - DNA, 19 bp, Artificial Sequence
  Edit genome.fa reference GFP10 (with barcode sequence 10)
  misc_feature (12)..(19): Barcode sequence 10
taaatcgatcgtgtgacca

SEQ ID NO: 27 - DNA, 19 bp, Artificial Sequence
  Edit genome.fa reference GFP11 (with barcode sequence 11)
  misc_feature (12)..(19): Barcode sequence 11
taaatcgatcgagggtcaa

SEQ ID NO: 28 - DNA, 19 bp, Artificial Sequence
  Edit genome.fa reference GFP12 (with barcode sequence 12)
  misc_feature (12)..(19): Barcode sequence 12
taaatcgatcgaggagtgg

SEQ ID NO: 29 - DNA, 5413 bp, Artificial Sequence
  pZac-CASI-GFP2-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 2
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgacagtggtaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 30 - DNA, 5413 bp, Artificial Sequence
  pZac-CASI-GFP3-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 3
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgcagatccaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 31 - DNA, 5413 bp, Artificial Sequence
  pZac-CASI-GFP4-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 4
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgacaaacggaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 32 - DNA, 5413 bp, Artificial Sequence
  pZac-CASI-GFP5-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 5
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgacccagcaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 33 - DNA, 5413 bp, Artificial Sequence
  pZac-CASI-GFP6-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 6
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgaacccctcaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 34 - DNA, 5413 bp, Artificial Sequence
  pZac-CASI-GFP7-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 7
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgcccaacctaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 35 - DNA, 5413 bp, Artificial Sequence
  pZac-CASI-GFP8-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 8
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgcaccacacaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 36 - DNA, 5413 bp, Artificial Sequence
  pZac-CASI-GFP9-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 9
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcggaaacccaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 37 - DNA, 5413 bp, Artificial Sequence
  pZac-CASI-GFP10-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 10
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgtgtgaccaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 38 - DNA, 5413 bp, Artificial Sequence
  pZac-CASI-GFP11-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 11
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgagggtcaaaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 39 - DNA, 5413 bp, Artificial Sequence
  pZac-CASI-GFP12-PolyA
  misc_feature (1)..(130): ITR sequence
  promoter (197)..(1252): CASI promoter sequence
  misc_feature (1268)..(1987): eGFP gene sequence
  misc_feature (2002)..(2009): AAV Barcode 12
  misc_feature (2016)..(2402): Polyadenylation tail sequence
  misc_feature (2462)..(2602): ITR sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataaatcgatcgaggagtggaccggttggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggatccgaattcgcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcctaattaaggccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 40 - DNA, 22 bp, Artificial Sequence
  Short sequence after the eGFP transgene stop codon in the plasmid backbone pZac2.1-CMV-eGFP.rgb for constructing barcoded eGFP plasmids
  misc_feature (15)..(22): n is a, c, g, or t
taataaatcgatcgnnnnnnnn

SEQ ID NO: 41 - DNA, 56 bp, Artificial Sequence
  GFP_NGS_P7Amp primer
gtgactggagttcagacgtgtgctcttccgatctgggcatggacgagctgtacaag

SEQ ID NO: 42 - DNA, 66 bp, Artificial Sequence
  GFP_NGS_P5Amp primer
acactctttccctacacgacgctcttccgatctgcaatgaaaataaatttcctttattagccaacc

SEQ ID NO: 43 - DNA, 58 bp, Artificial Sequence
  P5 Universal Primer
aatgatacggcgaccaccgagatctacactctttccctacacgacgctcttccgatct

SEQ ID NO: 44 - DNA, 66 bp, Artificial Sequence
  P7 Barcode Adapter_UDI0001 primer
caagcagaagacggcatacgagatagcgctaggtgactggagttcagacgtgtgctcttccgatct

(19) United States
(12) Patent Application Publication
(10) Pub. No.: US 2023/0257736 A1
(43) Pub. Date: Aug. 17, 2023

(54) A Method for Assessing Transduction Efficiency and/or Specificity of Vectors at Single Cell Level

(71) Applicant: AGENCY FOR SCIENCE, TECHNOLOGY AND RESEARCH, Singapore (SG)

(72) Inventors: Wei Leong Chew, Singapore (SG); Choong Tat Keng, Singapore (SG); Ke Guo, Singapore (SG); Daryl Lim, Singapore (SG)

(21) Appl. No.: 18/009,377

(22) PCT Filed: Jun. 11, 2021
(86) PCT No.: PCT/SG2021/050338

§ 371 Date: Dec. 9, 2022

(30) Foreign Application Priority Data
Jun. 12, 2020 (SG) 10202005599R

(51) Int. Cl. C12N 15/10

(52) U.S. Cl. CPC C12N 15/1082; C12N 15/1065

(57) ABSTRACT

Disclosed is a method for assessing the transduction efficiency and/or specificity of vectors at single cell level.

### CROSS-REFERENCE TO RELATED APPLICATIONS

This application claims the benefit of priority of Singapore patent application No. 10202005599R, filed 12 Jun. 2020, the contents of it being hereby incorporated by reference in its entirety for all purposes.

### FIELD OF THE INVENTION

The present invention generally relates to molecular biology and genomics. In particular, the present invention relates to a method for assessing vector transduction.

### BACKGROUND OF THE INVENTION

Since the discovery of methods that allow the genetic modification of cells, gene therapy has been touted to be one of the most promising modes of disease treatments. Gene therapy involves the alteration of the genes, usually defective or aberrant genes, in the cells of the subject to treat or prevent the disease. This is usually done by the introduction of a normal gene into the cell. The introduction of the normal gene can be done by the use of delivery vectors, wherein the transduction efficiency and transduction specificity of the delivery vectors are important features to allow for improved success of gene therapy.

Although different gene therapy methods have been widely studied, there is currently a gap in technology for high throughput studies or screening of libraries or panels of delivery vectors, such as naturally occurring or recombinant Adeno-associated virus (AAV) serotypes, for assessing the efficiency and specificity or tropism each of these vectors deliver to an individual cell type. This is made even more impractical or impossible when the cellular composition is heterogeneous comprising of many different cell types, for example in human tissues or within multiple organs in the human body.

In view of the above, as organs and tissues are generally heterogeneous, and the infection efficiency could vary considerably between niches within the organ/tissue, there is a need to provide a method for assessing the transduction efficiency and/or transduction specificity of viral vectors at a higher resolution, for example at a single cell level.

### SUMMARY OF THE INVENTION

In one aspect, there is provided a method for assessing the transduction efficiency and/or specificity of vectors at single cell level, said method comprising:
a) providing a plurality of different vectors,
b) transducing a heterogeneous population of cells with the plurality of different vectors;
c) partitioning the heterogeneous population of cells into a plurality of compartments, wherein each compartment comprises a single cell from the heterogeneous population of cells;
d) subjecting each partitioned cell to nucleotide sequencing;
e) detecting the presence of the any one or more of the different vectors in each partitioned cell.

### BRIEF DESCRIPTION OF THE DRAWINGS

The invention will be better understood with reference to the detailed description when considered in conjunction with the non-limiting examples and the accompanying drawings.

FIG. 1 is an exemplary schematic of the present disclosure: (A) A first example of an Adeno-associated virus (AAV) variant wherein the Adeno-associated virus (AAV) serotype variants are individually packaged with uniquely identifiable genomes, with the genomes each containing a unique nucleotide barcode. (B) A second example of an Adeno-associated virus (AAV) variant wherein the genomes each containing a unique capsid-encoding nucleotide sequence is located between the flanking Adeno-associated virus (AAV) inverted terminal repeats (ITRs). (C) A third example of an Adeno-associated virus (AAV) variant wherein the genomes, each containing a unique capsid-encoding nucleotide sequence between the flanking AAV ITRs, are packaged in multiplex based on capsid:genotype linkage, also known as genome:phenotype linkage, known in AAV biology. (D) Library of AAV variants from any one of (A) to (C) consisting a multitude of capsid serotypes, each identifiable by their encapsulated and uniquely differentiable nucleotide sequences. (E) Library of AAV variants is transduced into a heterogenous population of cells (e.g. organs, tissues, organoids, admixtures). (F) In single-cell sequencing, nucleic acids (which can include RNA, DNA) within each cell are tagged by a unique cell-specific single-cell-sequencing nucleotide tag and then sequenced. Each cell is identified by its RNA transcriptome and/or DNA genome. As the subset of AAV genomes that reside within each cell are tagged with the cell-specific single-cell-sequencing nucleotide tag, they can be identified by different methods. For example, the subset of AAV variants from (A) transduced into each cell are identified by their unique barcodes by short-read sequencing or long-read sequencing. The subset of AAV variants from (B) or (C) transduced into each cell are identified via unique capsid-encoding nucleotide sequences through the use of long-read sequencing, such as nanopore sequencing or single-molecule sequencing. (G) The matrix of the cell identity that is transduced by the specific AAV serotype can be created by matching the AAV variants with their respective transduced cells via the cell-specific single-cell-sequencing nucleotide tags.

FIG. 2A shows low magnification bright-field images of the gross morphology of developing human cerebral and ocular organoids that are cultured for 6 weeks. The organoids include fluid-filled cavities of ocular (white solid arrow) and solid brain (white dotted arrow) organoids. FIG. 2B shows images of histology sections of ocular organoids stained for cellular markers for cell-type characterization. S100β—neuronal crest and developed ocular: PAX6—ocular epithelial or endothelial cells; CHX10-specification and morphogenesis of the sensory retina; RAX—developing eye and initial specification of retinal cells; CD31—Schlemm's canal endothelial; aSMA—trabecular meshwork and stroma; DAPI (49, 6-diamidino-2-phenylindole) stain for nuclei; Neg—negative control. FIG. 2C shows images of histology sections of cerebral organoids sections stained with for cellular markers for cell-type characterization. MAP2—Positive in all neural cells; NeuN—Neuronal marker; S100β—detect brain proteins and express in the neuronal cells; DAPI—stain for nuclei. Thus, FIG. 2 illustrates the culture and characterization of ocular and cerebral organoids.

FIG. 3A shows low magnification microscopic images of the gross morphology of cerebral and ocular organoids infected with AAV serotypes pool, identified by the GFP-positive signals in cells within most regions. Barcoded GFP-AAV-Pool (1×10¹⁰ vector genomes (vg)/per serotype) expressing eGFP were used for transduction of cerebral and ocular organoids for 7 days. Mock indicates negative control of untransduced organoids. FIG. 3B shows cross-sectioned images of AAV-infected ocular organoid, wherein the AAV infection is identified by the GFP expression in different regions of the organoids. Image inserts represent regions with predominant cell-types: 1: corneal cell-types; 2: retinal cell-types; 3: neuronal cell-types. FIG. 3C shows images of immunofluorescence staining of cellular markers and GFP protein for identification of cell types transduced by the AAV serotypes pool. PAX6—ocular epithelial or endothelial cells; CHX10—specification and morphogenesis of the sensory retina; ZO-1—corneal endothelia marker. MAP2—neuronal marker; DAPI—stain for nuclei. Thus, FIG. 3 illustrates the characterization of pooled AAV infection of ocular and cerebral organoids, and an indication of high transduction efficiency of the pooled AAV serotypes.

FIG. 4A shows the schematic of general designs of the vectors as described herein. FIG. 4B shows the schematic of an exemplary design of AAV genomic cargo for capture and analysis of serotype barcodes. FIG. 4C shows the schematic of other exemplary designs of AAV with transgene encoding for the AAV viral capsid protein, which in turn encapsulates its self-encoding transgene. FIG. 4D shows the schematic of design of an exemplary AAV genomic cargo for capture and analysis of serotype barcodes. A mammalian promoter is selected for expression of a non-host protein in the human organoid cells. An eGFP transgene with barcode is expressed and can be distinguished from host gene transcripts. A unique 8 base-pair barcodes is included after the stop codon and before the polyadenylation tail, designed to be within the 98 bases from captured tail for Cell Ranger analysis. A polyadenylation tail sequence included for captured of RNA transcripts to the probes on 10× beads. FIG. 4E shows the modification of 10× Cell Ranger pipeline to include captured AAV serotype barcodes for high-throughput tropism analysis. The underlined sequences in SEQ ID NOs: 17-28 represent exemplary barcode sequences. Thus, FIG. 4 illustrates the exemplary vector design, for example, of AAV genomic cargo sequence for serotype barcoding and RNA transcripts capture, and modification of 10× Cell Ranger pipeline for high-throughput single-cell analysis of AAV tropism.

FIG. 5 shows one image, one heatmap and one table. FIG. 5A shows the t-Stochastic Neighbor Embedding (t-SNE) plot of 5849 cells from human ocular organoids derived from H1 human ES cells separated into 10 distinct clusters by K-means. Cluster 9 and 10 with low cell numbers were removed from subsequent AAV tropism analysis. The sequenced FASTQ files are processed by a modified Cell Ranger pipeline and visualized on the Cell Loupe software, the mean reads per cell is 122688 and the median genes per cell is 1022. FIG. 5B shows a representative list of top 10 high-expressing genes for each cell cluster as used for identification of cell niche in the t-SNE plot. Thus, FIG. 5 illustrates the use of single-cell RNA transcriptome analysis and niche markers identification for ocular organoids.

FIG. 6 shows nine images, two donut maps, 2 column graphs and 1 heatmap. FIG. 6A shows nine t-SNE plots showing individual cells transduced with different AAV serotypes (AAV1, AAV2, AAV6, AAV7, AAV8, AAV9, AAVrh10, AAV-DJ and AAV-Anc80), wherein each serotype is represented by one plot. Each plot comprises 10 clusters of the ocular organoid. The areas represented by the dark gray dots represent single cells that were successfully transduced by the particular AAV serotype, as determined by barcode counts within the cell. FIG. 6B shows the graphs representing bulk analysis of the transduced ocular organoid by amplicon-sequencing on MiSeq sequencer. Results of pre-infection and post-infection AAV from bulk sequencing analysis is in agreement with the single-cell analysis plots processed with Cell Ranger pipeline, indicating that this assay enables accurate measurement of AAV tropism with single-cell resolution, beyond traditional bulk sequencing approach. FIG. 6C shows the graph of counts of cells that are transduced with each AAV serotype in each cluster. Data showed unique transduction level of each AAV serotype across the different cell clusters within human ocular organoids. FIG. 6D shows the graph of AAV cell cluster tropism in transduced human ocular organoid. Results demonstrated that the tropism of each AAV serotype varied across the different cell clusters and are distinct from other AAV serotypes. FIG. 6E shows the transduction efficiency of each AAV serotype for each cell cluster is visualized as the percentage of cells transduced in a heat map plot. Using this method, it enables the identification of (i) the most efficient AAV serotype for each cell cluster; and (ii) the most specific AAV serotype for the target cell type of choice (i.e. lowest transduction of other non-desired cell types). Thus, FIG. 6 illustrates the use of high-throughput AAV tropism measurement and analysis for human ocular organoids.

FIG. 7 shows one image, one heatmap and one table. FIG. 7A shows the t-Stochastic Neighbor Embedding (t-SNE) plot of 15466 cells from human cerebral organoids derived from H1 human embryonic stem cells, separated into 10 distinct clusters by K-means. The sequenced FASTQ files are processed by a modified Cell Ranger pipeline and visualized on the Cell Loupe software, the mean reads per cell is 23315 and median genes per cell is 902. FIG. 7B shows a representative list of top 10 highly-expressing genes for each cell cluster for identification of the cell niche in the t-SNE plot. Thus, FIG. 7 illustrates the use of single-cell RNA transcriptome analysis and cell niche markers identification for human cerebral organoids.

FIG. 8 shows nine images, two donut maps, 2 column graphs and 1 heatmap. FIG. 8A shows nine t-SNE plots showing individual cells transduced with different AAV serotypes (AAV1, AAV2, AAV6, AAV7, AAV8, AAV9, AAVrh10, AAV-DJ and AAV-Anc80), wherein each serotype is represented by one plot. Each plot comprises 10 clusters of the cerebral organoid. The areas represented by the dark gray dots represent single cells that were successfully transduced by the particular AAV serotype, as determined by barcode counts within the cell. The bulk analysis of transduced cerebral organoid by amplicon-sequencing on MiSeq sequencer. FIG. 8B shows the graphs representing bulk sequencing analysis using custom Python script is in agreement with the single-cell analysis plots processed with Cell Ranger pipeline, indicating that this invention enables accurate measurement of AAV tropism with single-cell resolution, beyond traditional bulk sequencing approach. FIG. 8C shows the graph of counts of cells that are transduced with each AAV serotype in each cluster. Data showed unique transduction level of each AAV serotype across the different cell clusters within human cerebral organoids. FIG. 8D shows the graph of AAV cell cluster tropism in transduced human cerebral organoid. Results demonstrated that the tropism of each AAV serotype varied across the different cell clusters and are distinct from other AAV serotypes. FIG. 8E shows the transduction efficiency of each AAV serotype for each cell cluster is visualized as the percentage of cells transduced in a heat map plot. Using this method, it enables identification of (i) the most efficient AAV serotype for each cell cluster and (ii) the most specific AAV serotype for the target cell type of choice (i.e. lowest transduction of other non-desired cell types). Thus, FIG. 8 illustrates the use of high-throughput AAV tropism measurement and analysis for human cerebral organoids.

### DETAILED DESCRIPTION OF THE PRESENT INVENTION

With the discovery of science and medicine, humans have been on a quest to find treatments for different diseases. Despite the rapid development of science and medicine over the last century, there are still many diseases that cannot be treated. These diseases usually belong to a group known as genetic diseases, which are exemplified by sickle cell disease and Huntington's disease.

Gene therapy was introduced to be a promising mode of disease treatments, including treatment of genetic diseases. One common method of gene therapy is the introduction of normal genes by delivery vectors into a subject to allow the production of the normal proteins to cure or prevent the onset of the disease. There are two key properties for therapeutic delivery vectors to improve the success rate of gene therapy: firstly transduction efficiency, or how well the vector delivers therapeutic cargo to desired target cells; and secondly transduction specificity, or how well the vector avoids off-target delivery into the other cells within the body. Traditionally, vectors such as individual adeno-associated virus (AAV) serotypes are individually administered to the cells/tissue/animal, following which bulk sampling from whole tissues are used to determine if the individual AAVs got into the tissue.

Adeno-associated viruses (AAVs) are medically and commercially attractive gene delivery vectors due to the recent successes in FDA and EMA approvals for AAV-based gene therapies, as exemplified by Glybera for the treatment of lipoprotein lipase deficiency, Luxturna for the treatment of inherited retinal disease, and Zolgensma for the treatment of paediatric spinal muscular dystrophy. The therapeutic application of AAV span from targeting small tissues in the eye to systemic distribution in the muscles as well as difficult-to-access systems such as the nervous system and vasculature. This versatility is enabled by the ability to manipulate the AAV protein capsid sequence, which in turns changes the serotype and confers preferential tropism towards desired tissues. The terms “tropism” or “viral tropism” as used herein refers to the ability and specificity of a given virus to infect a cell type, tissue or species. While considerable efforts have been devoted to identifying optimal capsid proteins for successful therapy, early studies comparing the performance of different AAV serotypes are often of low-throughput and costly.

A first limitation is that each cell line or animal is usually only transduced by a single AAV serotype and hence to evaluate multiple different serotypes would require a similar increase in independent replicates; this is in part because readouts employed for transduction efficiency assays tend to be non-multiplexable, such as quantification by immunohistology or fluorescence reporter proxy, which means that each sample could only be treated by a single vector test candidate. A second limitation is that the sensitivity of transduction assays tends to require aggregation across many cells and vector copies, and hence the resolution is limited to the tissue level instead of the often required cellular level. Such single-plex approaches limit comparison to only a small handful of AAV serotypes in a similarly small number of target cells or tissues. In recent years, transduction assays of higher throughput have been devised by harnessing sequencing as a readout for transduction efficacy, whereby multiplex libraries of AAVs bearing nucleotide barcodes are administered to the target cells or tissues and the best-performing AAV serotype are identified by sequencing the nucleotide barcodes. However, the techniques employed so far have been limited to bulk tissues, which do not offer the resolution needed to profile how efficiently or specifically each AAV serotype transduces specific subset(s) of cells within a complex tissue population.

This tissue level analysis is insufficient if the desired target is, for example, a cell niche within the complex tissue in comparison to the other cell types within the tissue. The result for the target would be inaccurate as the readout would be diluted in the readout of the other cell types within the tissue. In addition, only a few serotypes would be assessed due to the difficulty in scaling up the traditional methods, wherein each ‘test’ includes separate administration of individual serotypes per sample, separate tissue section staining, and separate serotype reporter detection assay, which is laborious and time consuming. The commonly used method of identification of the different cell niches/types within the tissue by immuno-histochemistry is also limited by the availability of cell type-specific antibodies marker. Therein lies the following problems: that the more efficient vector might not be chosen for therapeutic use; the vector specificity is not known; it is not feasible to test many existing vectors; the occurrence of false positives, when target cells are not transduced but the neighboring cells are in bulk tissues; and the occurrence of false negatives, when target cells are transduced but the neighboring cells are not in bulk tissues.

In view of the above problems, there is a need to provide a novel method that enables multiplex measurement of transduction efficiency and/or transduction specificity. In particular, the method can measure how libraries of delivery vectors, for example but not limited to adeno-associated viral (AAV) and variants thereof, deliver into libraries of diverse cell types, for example but not limited to human cells in organoid cultures. In an exemplary method, it is demonstrated that pairing high-throughput measurement of AAV identity biodistribution with high-resolution single-cell RNA transcriptomic sequencing enables the unprecedented mapping of how natural and engineered AAV variants transduce human cells within cerebral and ocular organoids. The method as disclosed herein can also be applied to the determination of safety and efficacy of therapeutic delivery vectors, thereby allowing for the successful approval and commercialization of therapeutic modalities.

The inventors of the present disclosure have found a method for assessing the transduction efficiency and/or specificity of viral vectors at single cell level, said method comprising:
a) providing a plurality of different viral vectors,
b) transducing a heterogeneous population of cells with the plurality of different viral vectors;
c) partitioning the heterogeneous population of cells into a plurality of compartments, wherein each compartment comprises a single cell from the heterogeneous population of cells;
d) subjecting each partitioned cell to nucleotide sequencing;
e) detecting the presence of the any one or more of the different viral vectors in each partitioned cell.

The terms “transduction” or “transducing” as used herein refer to the process that a polynucleotide or nucleic acid can be introduced into a host cell. The polynucleotide or nucleic acid can be, but is not limited to vectors, DNA, RNA or plasmids. Therefore, the term “transduction efficiency” as used herein refers to the ability that a polynucleotide or nucleic acid can be introduced into a host cell by the vector. In one example, the transduction efficiency of a specific vector against a specific cell type is determined by the percentage of cells of the specific cell type which have been detected positive for the presence of the specific vector. In another example, the transduction efficiency of a specific vector against a specific cell type is assessed by comparing the frequencies with which the presence of said specific vector is detected in the cells of said specific cell type, against the frequencies with which the presence of another vector is detected in the cells of said specific cell type.

The term “transduction specificity” as used herein refers to the ability of the vector in transducing the target cells, or how well the vector avoids off-target delivery into the other cells within the body. In one example, the transduction specificity of a specific vector against a specific cell type relative to another cell type is assessed by the comparing the frequencies with which the presence of said specific vector is detected in the cells of said specific cell type, against the frequencies with which the presence of said specific vector is detected in the cells of another specific cell type.

The term “vector” as used herein refers to a macromolecule or association of macromolecules that comprises or associates with a polynucleotide and which can be used to mediate delivery of the polynucleotide to a cell. Exemplary vectors include, but are not limited to, plasmids, viral vectors (virus or the viral genome thereof), pseudo-virus vectors, virus-like particles, liposomes, exosomes, nanoparticles, and other gene delivery vehicles. In one example, the vectors are selected from the group consisting of: a viral vector, a pseudo-virus vector, a virus-like particle vector, a liposome vector, an exosome vector, a nanoparticle, and combinations thereof; wherein the vectors comprise DNA, RNA, modified RNA, modified DNA, or combinations thereof.

In one example, the vectors comprise viral vectors, wherein the viral vectors are selected from the group consisting of: an adenoviral vector, an Adeno-associated virus (AAV) vector, a lentiviral vector, a coronavirus vector, an enterovirus vector, a retroviral vector, or a combination thereof. In another example, the plurality of different viral vectors comprises viral vectors of different families, viral vectors of different genera, viral vectors of different species, viral vectors of different serotypes, viral vectors thereof carrying different mutations, or combinations thereof. In a preferred example, the viral vectors are AAV vectors. In a further example, the viral vectors are selected from the group consisting of: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV9), AAV type 10 (AAV10), AAV type 11 (AAV11), AAV type 12 (AAV12), AAV type 13 (AAV13), rh10, AAVDJ, AAVAnc80, AAV-PHP.S, AAV-PHP.eB, AAV-LK03, AAV2-7m8, AAV variants thereof, and combinations thereof. The term “AAV variant” includes an AAV viral particle comprising a variant, or mutant, AAV capsid protein. Examples of variant AAV capsid proteins include AAV capsid proteins comprising at least one amino acid difference (e.g., amino acid substitution, amino acid insertion, amino acid deletion) relative to the capsid protein of a corresponding parental AAV (or a AAV serotype).

In the method as disclosed herein, each of the plurality of different vectors comprises an oligonucleotide barcode sequence, wherein the barcode sequence is different between any two different vectors. The term “barcode,” as used herein, generally refers to a label, or identifier, that can be part of an analyte to convey information about the analyte. A barcode can be a tag attached to an analyte (e.g., nucleic acid molecule) or a combination of the tag in addition to an endogenous characteristic of the analyte (e.g., size of the analyte or end sequence(s)). In one example the barcode is unique. Barcodes can have a variety of different formats, for example, barcodes can include, but are not limited to: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. In one example, the barcode sequence is located on an expression cassette in the vector, wherein the expression of the cassette results in the production of an RNA molecule comprising the barcode sequence, wherein the RNA molecule further comprises a polyadenylation tail.

In another example, the barcode sequence is located on a region of the RNA molecule which allows the barcode sequence to be sequenced. The region of the RNA molecule which allows the barcode sequence to be sequenced can be in proximity to the polyadenylation tail or is distant from the polyadenylation tail. The term “polyadenylation tail” as used herein refers to a stretch of RNA that has only adenine bases. The barcode sequence can be within a distance of 1 to 100 nucleotides from the polyadenylation tail. In one example, the barcode sequence can be within a distance of 1 to 10 nucleotides, 11 to 20 nucleotides, 21 to 30 nucleotides, 31 to 40 nucleotides, 41 to 50 nucleotides, 51 to 60 nucleotides, 61 to 70 nucleotides, 71 to 80 nucleotides, 81 to 90 nucleotides, or 91 to 100 nucleotides from the polyadenylation tail. In one example, the barcode sequence can be within a distance of 91, 92, 93, 94, 95, 96, 97, 98, 99 or 100 nucleotides from the polyadenylation tail. In a preferred example, the barcode sequence is within a distance of 98 nucleotides from the polyadenylation tail.

The barcode sequence can range from 1 to 100 nucleotides in length. In one example, the barcode sequence can be 1 to 10 nucleotides, 11 to 20 nucleotides, 21 to 30 nucleotides, 31 to 40 nucleotides, 41 to 50 nucleotides, 51 to 60 nucleotides, 61 to 70 nucleotides, 71 to 80 nucleotides, 81 to 90 nucleotides, or 91 to 100 nucleotides in length. In another example, the barcode sequence is 5, 6, 7, 8, 9, 10, 11, 12, 13, 14, 15, 16, 17, 18, 19, 20, 21, 22, 23, 24, 25, 26, 27, 28, 29, 30 nucleotides in length. In a preferred example, the barcode sequence is 8 nucleotides in length.

To aid in the detection of the barcode sequence, a tag sequence can be included beside or in close proximity to the barcode sequence. In one example, the tag sequence is upstream of the barcode sequence. In another example, the tag sequence is downstream of the barcode sequence. The tag sequence can be found within a distance of 1 to 10 nucleotides, 11 to 20 nucleotides, or 21 to 30 nucleotides from the barcode. In one example, the tag sequence can be found within a distance of 9, 10, 11, 12, 13, 14, 15, 16, 17, 18, 19 or 20 nucleotides from the barcode. In a preferred example, the tag sequence can be found within a distance of 15 nucleotides from the barcode.

The tag sequence encodes a detectable label for the purpose of detecting the barcode. Non-exhaustive examples of such tag sequence can encode for fluorescent proteins, epitopes, or any affinity tags. In one example, the tag sequence encodes for, but is not limited to, green fluorescent protein (GFP), red fluorescent protein (RFP), blue fluorescent protein (BFP), FLAG, HA, streptavidin or glutathione S-transferases (GST). In another example, the tag sequence encodes for green fluorescent protein (GFP). In another example, the tag sequence is SEQ ID NO: 9.

In the method as disclosed herein, the plurality of vectors can further comprise a marker polynucleotide. In one example, each of the plurality of different vectors comprises a marker polynucleotide, wherein the marker polynucleotide is different between any two different vectors; and wherein the marker polynucleotide encodes for one or more proteins, said one or more proteins when expressed form a protein envelope which encapsulate the marker polynucleotide, so that after transfection of the vector, each marker polynucleotide is encapsulated by the one or more proteins which the marker polynucleotide encodes for. In one example, the marker polynucleotide is located on an expression cassette in the vector, wherein the expression of the cassette results in the production of an RNA molecule comprising the marker polynucleotide, wherein the RNA molecule further comprises a polyadenylation tail. The marker polynucleotide can be, but is not limited to a gene encoding a portion of a virus. The portion of the virus can include a viral-capsid-encoding gene and/or a viral-replication gene, wherein the capsid expressed by the marker polynucleotide encapsulates the marker polynucleotide. In another example, the marker polynucleotide is a viral-capsid-encoding gene, wherein the capsid expressed by the marker polynucleotide encapsulates the marker polynucleotide. The marker polynucleotide that encodes the viral capsid proteins encapsulating itself is known as genotype:phenotype linkage, also known as capsid-genotype linkage. Genotype:phenotype linkage occurs in the viral production process when a multitude of AAV capsid transgene variants are introduced into the host cells, resulting in the production of a multitude of AAV capsid proteins. Each variant (or serotype) of AAV capsids then encapsulates the specific capsid transgene that has encoded these specific capsids. The multitude of AAV capsid variants may differ, but the transgene sequences, when translated, largely match to their respective encapsulating capsid protein sequences. In another example, the marker polynucleotide comprises SEQ ID NO: 12, SEQ ID NO: 13, SEQ ID NO: 14, SEQ ID NO: 15 or SEQ ID NO: 16. In one example, the viral-capsid-encoding gene is specifically an AAV-capsid-encoding gene. In another example, the viral-capsid-encoding gene is SEQ ID NO: 14, SEQ ID NO: 15 or SEQ ID NO: 16.

The vectors as described herein can further comprise a promoter sequence. The promoter sequence allows the binding of the RNA polymerase and transcription factors, therefore controlling the expression of the target gene. The promoter sequence can include, but are not limited to, P5, CASI, or cytomegalovirus (CMV) promoter sequence. In one example, the promoter sequence is a P5 promoter sequence. In another example, the promoter sequence is a CASI promoter sequence. In another example, the promoter sequence is SEQ ID NO: 8 or SEQ ID NO: 11.

The vectors as described herein can further comprise one or more inverted terminal repeats (ITRs). The inverted terminal repeats (ITRs) contain the origins of replication, which is a nucleotide sequence at which replication is initiated. In one example, the one or more inverted terminal repeats (ITRs) sequence is selected from the group consisting of SEQ ID NO: 5, SEQ ID NO: 6, and SEQ ID NO: 7.

The vectors as described herein are used to transduce a heterogenous population of cells. The term “heterogenous population of cells” as used herein refers to a group of cells that are dissimilar genetically, phenotypically or morphologically. The heterogenous population of cells can include different cell types from different organisms. In one example, the heterogeneous population of cells comprise plant cells, animal cells, fungal cells, or combinations thereof. The animal cells can be, but is not limited to mammalian, reptilian, insect or avian cells. In one example, the heterogeneous population of cells comprise mammalian cells. “Mammalian cells” include cells from humans and both domestic animals such as laboratory animals and household pets, (e.g. cats, dogs, swine, cattle, sheep, goats, horses, rabbits), and non-domestic animals such as wildlife, fowl, birds and the like. In a preferred example, the heterogeneous population of cells comprise human cells. The heterogenous population of cells can also include different cell types within the same or different tissues, organs or organoids. The heterogenous population of cells may include, but are not limited to endodermal cells, mesodermal cells, ectodermal cells, or any cell type that can be derived thereof. In another example, the heterogeneous population of cells are cultured cells. In another example, the heterogeneous population of cells are obtained from one or more cultured organoids. The term “organoid” refers to a cell cluster or aggregate that is considered a miniaturized and simplified version of an organ produced in vitro in three dimensions. Typically, an organoid shows realistic micro-anatomy that resembles an organ, or part of an organ, and possesses cell types relevant to that particular organ. In another example, the one or more cultured organoids are selected from the group consisting of ocular organoid, cerebral organoid, epithelial organoid, kidney organoid, lung organoid, pancreas organoid, cardiac organoid, and hepatic organoid. In a preferred example, the one or more cultured organoids are ocular organoid or cerebral organoid.

The method as described herein can apply in vivo, in vitro or ex vivo. In one example, the heterogeneous population of cells are comprised in an animal or human subject when being transduced.

The method as described herein can comprise further steps. In one example, the method further comprises:
f) classifying each partitioned cell into a specific cell type based on gene expression patterns and/or epigenetic features of said cell, as determined using sequencing results obtained in step d).

As used herein, the term “epigenetic” describes the state or condition of DNA with respect to changes in function without a change in the nucleotide sequence. Examples of epigenetic features, which may be naturally present or results of modification, include but are not limited to DNA methylation, histone modification, chromatin accessibility, sites of nucleosomes and nucleosome-free regions, etc. The epigenetic features may lead to changes in the expression of genes.

The method as described herein can comprise further details within any one of steps a) to f). In one example, step e) comprises detecting the presence of one or more marker sequences specific to each different vector; wherein when each vector comprises a unique barcode sequence, said one or more marker sequences comprise the barcode sequence; wherein when each vector comprises a unique marker polynucleotide, said one or more marker sequences comprise the marker polynucleotide. In another example, step e) comprises matching the sequence reads obtained in step d) with a reference data set. The reference data set comprises the genomes and/or the transcriptomes of the plurality of different viral vectors, and/or the barcodes comprised in the plurality of different viral vectors, and/or the marker polynucleotides comprised in the plurality of different viral vectors.

To achieve the partitioning of the heterologous population of cells into different compartments, different methods can be used. The terms “partitioning” or “partitioned” as used herein refer to the separation of the cells into different sections or compartments. In a preferred example, the compartments are oil droplets.

The nucleotide sequencing as described in the method herein can be any sequencing method that is generally known in the art. In one example, the nucleotide sequencing is RNA sequencing. In another example, the nucleotide sequencing is DNA sequencing.

Various methods can be used to detect the presence of any one or more of the different viral vectors in each partitioned cells. In one example, the method to detect the presence of any one or more of the different viral vectors in each partitioned cells includes, but is not limited to sequencing, multiplex qPCR, in situ sequencing or in situ hybridization. In a preferred example, the method to detect the presence of any one or more of the different viral vectors in each partitioned cells is sequencing.

The present disclosure provides for a method that allows high-throughput identification of efficiency, biodistribution and cell/tissue-type specificity of delivery vectors, for example but not limited to a multitude of recombinant adeno-associated viruses (rAAVs), nucleic acids, viruses, nanoparticles, liposomes, or purified biomolecules, at single-cell resolution within heterogenous and/or complex cell populations. Examples of heterogenous and/or complex cell populations can be, but are not limited to human organoids, human tumor, human biopsy, human tissue, human organ, mixture of human cells, plant tissue, animal tissue, or mixture of cells. The method as described herein enables high-throughput determination of vector efficiency and specificity in conditions including a multiplex (i.e. multiple tests simultaneously in the same experiment/sample) setup, at single-cell resolution, within complex tissues without the need for any pre-enrichment, or within any cell types of interest. The method as described herein also provides for the identification of the most efficient delivery vector composition for targeted nucleic acid delivery to single cells or single-cell niche clusters of interest. This is achieved by comparative identification and analysis of the frequencies that individual composition sequences are found in each cell or cell niche cluster. The method as described herein can also provide for determining the specificity and efficiency of different AAV serotype(s) across single cells or single-cell niches in the human tissue.

In summary, the present disclosure provides the following:
A. A demonstration of single-cell sequencing for identifying the tropism (specificity) of multiple AAVs simultaneously, at single-cell level in complex cell population.
B. A method of indexing individual AAVs via unique sequences (indexes) and integrating the capture process and sequencing of the indexes into the experimental and bioinformatics workflow for single-cell transcriptomic profiling that can determine the cell types. The unique sequences can be, for example, an 8-base DNA per serotype, or capsid-encoding DNA sequences. Such a method allows the identification of frequencies of the multiple or single AAVs in each unique cell type.
C. A method of performing comparative AAV transduction efficiency and specificity in the heterogeneous cell populations.

The method as disclosed herein allows sequence alignment specifically to various non-host AAV serotypes sequence and assign them to single cells niche based on their RNA expression profile. Presently, the commonly used ubiquitous single cell technology is designed to only align only to human RNA transcriptome to achieve an RNA expression profile for tissues. The method as disclosed herein is also made possible by the incorporation of a polyadenylation tail in the vector, which is normally not present in virus proteins. The incorporation of the polyadenylation tail enables the capture of the expressed proteins. In one example, the polyadenylation tail sequence is SEQ ID NO: 10. This is coupled with the incorporation of unique barcodes for each AAV serotype within the sequence region upon capture alongside the human RNA transcriptomes. The method also includes the modification of the reference data set as well as the modification of the alignment commands, which allows for the final data to be extracted and analyzed for the transduction efficiency and transduction specificity.

As used in this application, the singular form “a,” “an,” and “the” include plural references unless the context clearly dictates otherwise. For example, the term “a genetic marker” includes a plurality of genetic markers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Material and Methods

Organoids Culture and Condition

Briefly, the cerebral and ocular organoids were cultured in mTeSR1 medium (Stem Cell Technologies, cat. no. 85850). Human ES cell (H1 WA01 and H9 WA09) were treated by accutase to generate single cells. In total, 4000 cells were plated in each well of a 96-well V Bottom plate (Sematec Pte Ltd Code: 1009985) with low concentration basic fibroblast growth factor (bFGF 4 ng/ml) and 20 uM/ml Rho-associated protein kinase (ROCK) inhibitor (Y27632 Stem Cell). For the culture of cerebral organoid, Embryonic Bodies (EB) were transferred after 24 hours to a low-attachment 96-well U-bottom plate with hESC medium (400 ml of DMEM-F12, 100 ml of KOSR, 15 ml of ESC-quality fetal bovine serum (FBS), 5 ml of GlutaMAX, 5 ml of MEM-NEAA and 3.5 μl of 2-mercaptoethanol). For the culture of ocular organoid, Embryonic Bodies (EB) were transferred after 24 hours to a low-attachment 96-well U-bottom plate with Differentiation Medium DM (DMEM/F12, 4% knockout serum replacement (KOSR), 4% ESC-quality fetal bovine serum (FBS), lx non-essential amino acids (NEAA), 1× Glutamax, 1× Pen-Strep; filtered using a vacuum-driven 0.2-μm filter unit). EB were fed every other day for 6 days before placing them into neural induction media for cerebral organoid and into retinal differentiation medium (RDM: DM+2% B27) for ocular organoid for the next 4 days. When the EB exhibited neuro-ectodermal differentiation (in 10 days), the aggregates were transferred to Matrigel (Growth factor-reduced Matrigel, Bio-Lab 354230). The Matrigel were made with cerebral organoid differentiation medium (CDM) in a 1:1 dilution ratio. 50 ul Matrigel was added into each well and incubated for 30 minutes at 37° C. in an incubator. 100 ul cerebral organoid differentiation medium with B27 (−) Vitamin A were then added to each well and cultured for 48 hours. After culturing for 2-3 days, the aggregates (organoids) were transferred to a 6-well clear flat bottom ultra-low attachment plate. After 4 days of static culture with cerebral organoid differentiation medium with B27 (−) Vitamin A, the embedded organoids were transferred to an orbital shaker at 80 rpm and placed in a 37° C., 5% CO2 incubator for long term culture (1 to 52 weeks) in cerebral organoid differentiation medium with B27 (+) Vitamin A.

AAV Plasmid Cloning and Virus Production

The barcoded eGFP plasmids were constructed by introducing a short sequence TAATAAATCGATCGNNNNNNNN (SEQ ID NO: 40) after the eGFP transgene stop codon in the plasmid backbone pZac2.1-CMV-eGFP.rgb. Primers with overhanging barcode were designed for first round PCR to generate barcoded eGFP fragments that terminates at ITR sequences. A second round of nested PCR amplify shorter fragments of barcoded eGFP which are digested with restriction enzymes NheI and BamHI. Digested fragments are ligated with the vector backbone, which are digested using the same restriction enzymes NheI and BamHI. The sequences of the clones were checked by Sanger sequencing. The representative barcodes for each AAV serotype are shown in Table 1.

TABLE 1

| Plasmids | Barcode | For barcoding |
|---|---|---|
| pZac2.1-CMV-eGFP_A701 | ATCACGAC | AAV1 |
| pZac2.1-CMV-eGFP_A702 | ACAGTGGT | AAV2 |
| pZac2.1-CMV-eGFP_A706 | AACCCCTC | AAV6 |
| pZac2.1-CMV-eGFP_A707 | CCCAACCT | AAV7 |
| pZac2.1-CMV-eGFP_A708 | CACCACAC | AAV8 |
| pZac2.1-CMV-eGFP_A709 | GAAACCCA | AAV9 |
| pZac2.1-CMV-eGFP_A710 | TGTGACCA | AAV-rh10 |
| pZac2.1-CMV-eGFP_A711 | AGGGTCAA | AAV-DJ |
| pZac2.1-CMV-eGFP_A712 | AGGAGTGG | AAV-Anc80 |

The serotype-specific pAAV-RepCap plasmids were constructed by cloning in the Cap genes from the different serotypes into the pAAV-RepCap backbone using Gibson assembly. The different serotypes Cap genes were ordered as gene blocks (IDT) and cloned into HindIII/PmeI-digested pAAV-RepCap backbone via Gibson assembly to construct the pAAV-RepCap with the different serotypes Cap genes. AAV viruses from different serotypes each bearing its own barcode were produced. Briefly, AAV were packaged via a triple transfection of 293AAV cell line (Cell Biolabs AAV-100) that were plated in a HYPERFlask ‘M’ (Corning) in growth media consisting of DMEM, glutaMax, pyruvate, 10% FBS (Thermo Fisher), supplemented with 1×MEM non-essential amino acids (Gibco). Confluency at transfection was between 70-90%. Media was replaced with fresh pre-warmed growth media before transfection. For each HYPERFlask ‘M’, 200 μg of pHelper (Cell Biolabs), 100 μg of pRepCap [encoding capsid proteins for different serotypes], and 100 μg of pZac-CASI-GFP (barcoded) were mixed in 5 ml of DMEM, and 2 mg of PEI “MAX” (Polysciences) (40 kDa, 1 mg/ml in H₂O, pH 7.1) added for PEI:DNA mass ratio of 5:1. The mixture was incubated for 15 minutes, and transferred drop-wise to the cell media. The day after transfection, media was changed to one consisting of DMEM, glutamax, pyruvate and 2% FBS. Cells were harvested 48-72 hours after transfection by scrapping or dissociation with 1× phosphate-buffered saline (PBS) (pH 7.2)+5 mM EDTA, and pelleted at 1500 g for 12 minutes. Cell pellets were resuspended in 1-5 ml of lysis buffer (Tris HCl pH 7.5+2 mM MgCl+150 mM NaCl), and freeze-thawed 3 times between dry-ice-ethanol bath and 37° C. water bath. Cell debris was clarified via 4000 g for 5 minutes, and the supernatant collected. The collected supernatant was treated with 50 U/ml of Benzonase (Sigma-Aldrich) and 1 U/ml of RNase cocktail (Invitrogen) for 30 minutes at 37° C. to remove unpackaged nucleic acids. After incubation, the lysate was loaded on top of a discontinuous density gradient consisting of 6 ml each of 15%, 25%, 40%, 60% Optiprep (Sigma-Aldrich) in a 29.9 ml Optiseal polypropylene tube (Beckman-Coulter). The tubes were ultra-centrifuged at 54000 rpm, at 18° C., for 1.5 hours, on a Type 70 Ti rotor. The 40% fraction was extracted and dialyzed with 1×PBS (pH 7.2) supplemented with 35 mM NaCl using Amicon Ultra-15 (100 kDa MWCO) (Millipore). The titer of the purified AAV vector stocks were determined using real-time qPCR with ITR-sequence-specific primers and probe26, referenced against the ATCC reference standard material 8 (ATCC).

In Vitro AAV Transduction of Organoids

AAV serotypes pool was created by pooling each AAV serotype at 1×10¹⁰ vector genomes (vg), giving a final viral copy of 9×10¹⁰ that is used for the transduction of organoids in each well of a 24-well plate. AAV1, 2, 6, 7, 8, 9, rh10, DJ and Anc80 serotypes were used for the pooling. Organoids were transduced for 7-10 days before harvesting for sequencing, fluorescence imaging, and histochemistry.

Immunofluorescence Histochemistry

Organoids were fixed in 4% paraformaldehyde for 4 hours at 4° C. followed by washing in PBS three times for 15 minutes. Organoids were allowed to sink in 30% sucrose overnight and then embedded in OCT and cryosectioned at 12 μm. Sections were permeabilized in 0.2% Triton X-100 in PBS and blocked using block buffer (2% bovine serum albumin (BSA) and 5% fetal bovine serum) for 1 hour at room temperature. Sections were subsequently incubated with the indicated primary antibodies at a 1:100 dilution in block buffer at 4° C. overnight. Secondary antibodies used were donkey Alexa Fluor 488, 568 and 647 conjugates (Invitrogen, 1:1000). After staining with 4′,6-diamidino-2-phenylindole (DAPI) (Sigma-Aldrich) in PBS for 5 minutes, slides were mounted in Vectashield anti-fade reagent (Vector Laboratories). Confocal imaging was performed with Leica TCS SP8 DLS LightSheet microscope. Primary antibodies: PAX6 (rabbit, abcam ab5790), CHX10 (rabbit, abcam ab133636), ZO-1 (mouse, Thermofisher ZO1-1A12), MAP2 (chicken, abcam ab5392). S100β (rabbit, abcam ab52642), RAX (rabbit, abcam ab23340), CD31 (mouse, abcam ab23340), aSMA (rabbit, abcam ab5694), DAPI (49,6-diamidino-2-phenylindole). NeuN (mouse, Sigma-Aldrich MAB377).

Design and Production of AAV Sequence

A mammalian promoter is selected for expression of a non-host protein in the human organoid cells. An eGFP transgene with barcode is expressed and can be distinguished from host gene transcripts. A unique 8 base-pair barcode is included after the stop codon and before the polyadenylation tail, and is designed to be within the 98 bases from captured tail for Cell Ranger analysis. A polyadenylation tail sequence is included for captured of RNA transcripts to the probes on 10× beads. Examples of the sequence of the plasmid include SEQ ID NOs: 1 and 29-39.

The transgene can also be designed to encode for the AAV viral capsid protein, which in turn encapsulates its self-encoding transgene. This is achieved by placing the AAV Rep and Cap-encoding sequences in between the AAV inverted terminal repeats (ITRs), for example, SEQ ID NO: 2 or SEQ ID NO: 3. This can also be achieved by placing the Cap-encoding sequence in between the AAV ITRs, as shown in SEQ ID NO: 4. The Cap sequences encode sequence variations on the nucleotide and encoded amino acid levels, differing by 1, 2, 3, 4, 5, 6, 7, 8, 9, 10, or more variations from each other.

The entire pool of such pAAV-Rep-Cap/pAAV-Cap variant plasmids is transfected into HEK293 cells for AAV production, together with the pHelper plasmid that encodes necessary adenoviral helper proteins. Individual AAVs produced from such a pooled format preferentially encapsulate their own encoding ITR-Rep-Cap-ITR or ITR-Cap-ITR cargoes. This is enabled by the genotype:phenotype linkage, also known as capsid:genotype linkage, in AAV packaging. The genotype:phenotype linkage is utilized as a way to package the library of AAVs in a way such that each AAV contains its own genome in a non-random fashion, therefore enabling the DNA/RNA sequencing to re-identify the protein capsid identities. The capsid sequence can be identified via long-read sequencing.

Amplicon Barcode Sequencing and Analysis

Transduced organoid samples were harvested as single cells and processed through the 10× chromium machine for cell barcoding of the transcripts. The total complementary DNA (cDNA) was purified via the 10× workflow and 5 ul was aliquoted for custom bulk-sequencing. The rest of the cDNA were used to proceed with the remaining 10× workflow for single-cell sequencing. Custom primers were designed for a first round of 20-cycle PCR of the target site containing the AAV barcodes as shown in Table 1. Target bands were extracted using gel extraction and a second round of 15-cycle PCR were used for adding P5 and P7 adapter sequences to the enriched fragments, and the final libraries were cleaned by gel extraction. Primers used for library construction are shown in Table 2. Library concentrations were determined using a Qubit dsDNA HS kit (Agilent). Next generation sequencing (NGS) sequencing was carried out on the MiSeq using 2×75 bp PE run with 20% PhiX spike-in. An in-house python script was utilized to search for the 8 unique nucleotide barcode sequences representing each serotype within the MiSeq FASTQs generated from the MiSeq run of the amplicon libraries and the total count was tabulated for each barcode sequence for each sample.

TABLE 2

| Primers Name | Sequence |
|---|---|
| GFP_NGS_P7Amp | GTGACTGGAGTTCAGACGTGTGCTCTTCCGATCTGGGCAT |
| | GGACGAGCTGTACAAG (SEQ ID NO: 41) |
| GFP_NGS_P5Amp | ACACTCTTTCCCTACACGACGCTCTTCCGATCTGCAATGA |
| | AAATAAATTTCCTTTATTAGCCAACC (SEQ ID NO: 42) |
| P5 Universal Primer | AATGATACGGCGACCACCGAGATCTACACTCTTTCCCTAC |
| | ACGACGCTCTTCCGATCT (SEQ ID NO: 43) |
| P7 Barcode | CAAGCAGAAGACGGCATACGAGATAGCGCTAGGTGACTG |
| Adapter_UDI0001 | GAGTTCAGACGTGTGCTCTTCCGATCT (SEQ ID NO: 44) |

Single Cell Sequencing and RNA Transcriptomic Analysis

Samples were prepared as indicated in the 10× Genomics Single Cell 3′ v2 Reagent Kit user guide. The single-cell libraries were prepared by following the manufacturers' protocol followed by sequencing on an Illumina HiSeq4000 flow cell. The sequencing data were processed by the standard Cell Ranger pipeline using the modified gtf and genome manifest files. Briefly, the samples were washed twice in PBS (Life Technologies)+0.04% BSA (Sigma) and re-suspended in the same solution. Sample viability was assessed using Trypan Blue (Thermo Fisher) under a light microscope. Following viability counting, the appropriate volume for each sample was calculated for a target capture of 10,000 cells and loaded onto the 10× Genomics single-cell-A chip along with other reagents and barcoded beads by following the protocol guide. The chip is then loaded onto a 10× Chromium machine for droplet generation and samples were transferred onto a pre-chilled strip tube (Eppendorf), and reverse transcription was performed using a 96-well thermal cycler (Thermo Fisher). After the reverse transcription, cDNA was recovered using Recovery Agent provided by 10× Genomics, followed by Silane DynaBead clean-up (10× Genomics). Purified cDNA was amplified for 12 cycles before being cleaned up using SPRI-select beads (Beckman). Samples were diluted 4 times in water and ran on a Bioanalyzer (Agilent Technologies) to determine cDNA concentration. cDNA libraries were then prepared following the Single Cell 3′ Reagent Kits v2 user guide with appropriate PCR cycles based on the cDNA concentration as determined by the bioanalyzer. The molarity of the single cell libraries was calculated based on their library sizes as measured using a bioanalyzer (Agilent Technologies) and using the KAPA qPCR quantification (KAPA) method on a qPCR cycler (Roche). Samples were normalized to 10 nM before sequencing. Each organoid sample was sequenced on a full lane on a HiSeq 4000 with the following run parameters: Read 1-26 cycles, read 2-98 cycles, index 1-8 cycles. Using the FASTQ files from each sample, the standard Cell Ranger Count command pipeline was performed for transcripts read alignment, UMI counting, and clustering (Amazon Web Services via the Ronin cloud platform).

The genome reference file was edited to include sequence of the barcoded eGFP for alignment. The gtf file is edited to include the barcoded eGFP transcripts into the transcriptome for reads counting and analysis. Raw data were processed using standard Cell Ranger transcriptomics command, while using modified genome reference file and the modified gtf file. Finally, the single-cell clusters and transcript counts were visualized in the Loupe Browser software user interface (10× Genomics).

Single Cell AAV Tropism Analysis

For parallel sequencing of the AAV barcodes in single cells along with the RNA transcripts, the human genome reference file and the genome transcript file (gtf) were modified. Briefly, the names and barcodes of each AAV serotypes are manually included into both files that will be used for the execution of the Cell Ranger Count command pipeline in order to include the AAV barcode transcripts into the read alignment, UMI counting, and clustering. To include the AAV barcode representation in the genome reference file, the command line “>GFP1 TAAATCGATCGNNNNNNNN” is included for each barcode, where the 8Ns represent a unique 8 nucleotide barcode sequence. The command line “GFP me exon 1 19—+—gene_id “GFP1”; transcript_id “GFP1”” was included in the genome transcript file for each AAV barcode representation added to the genome reference file.

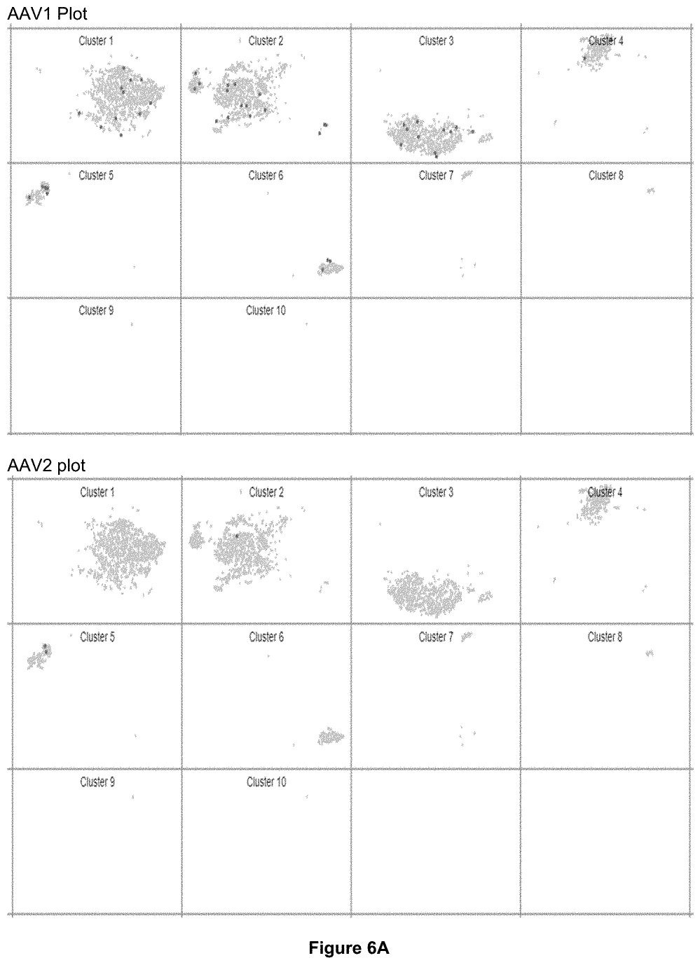

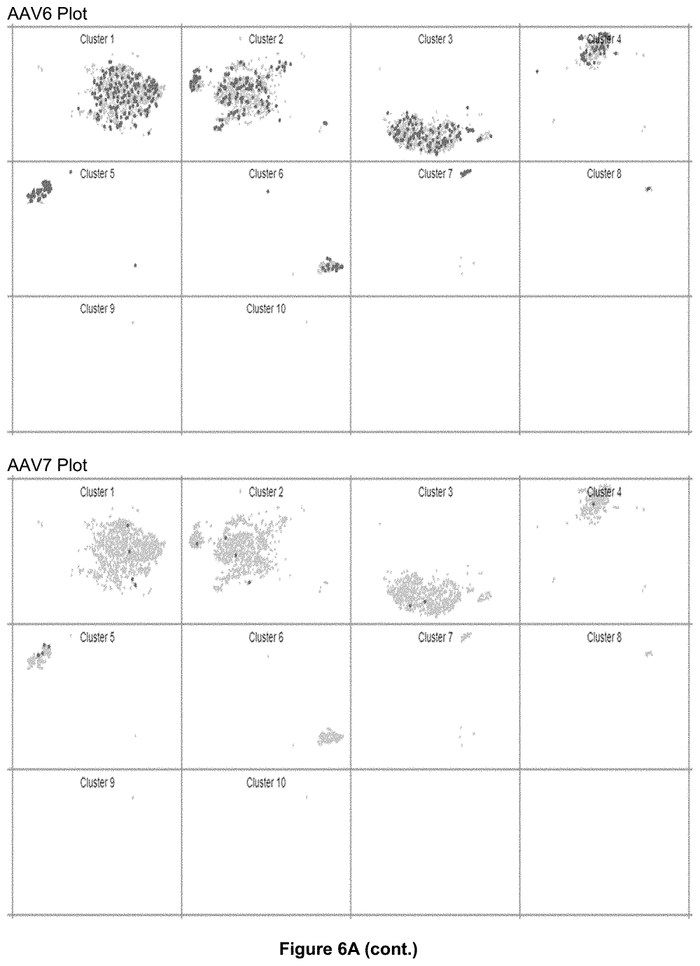

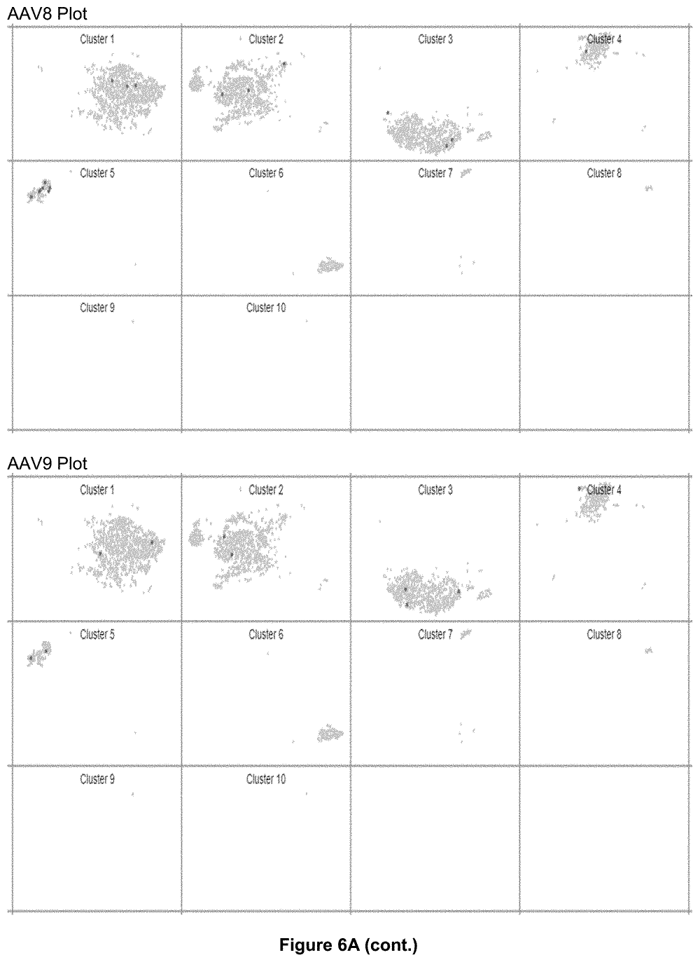

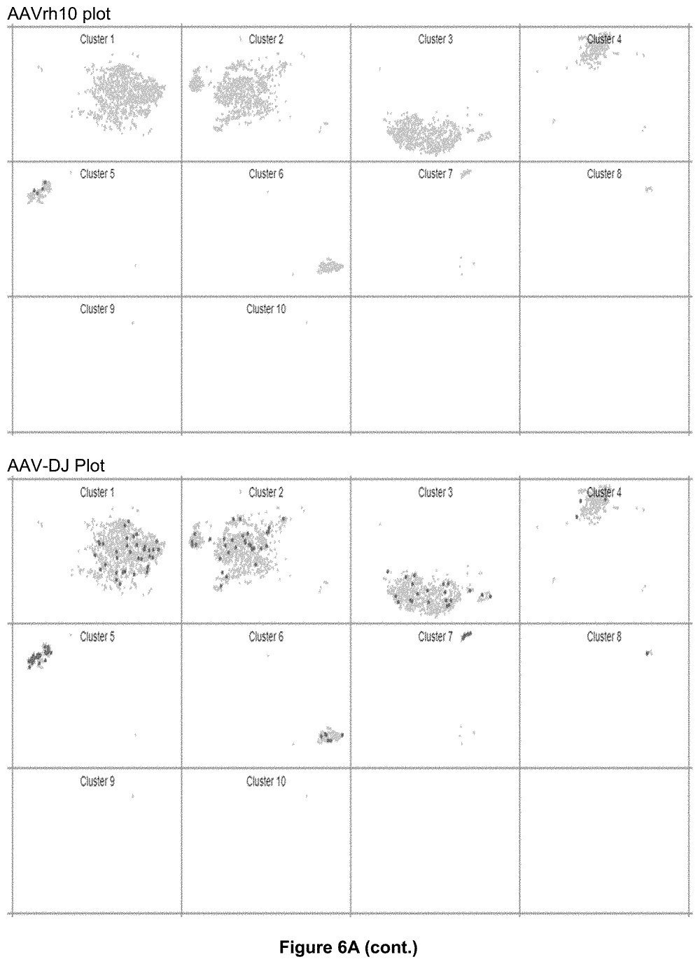

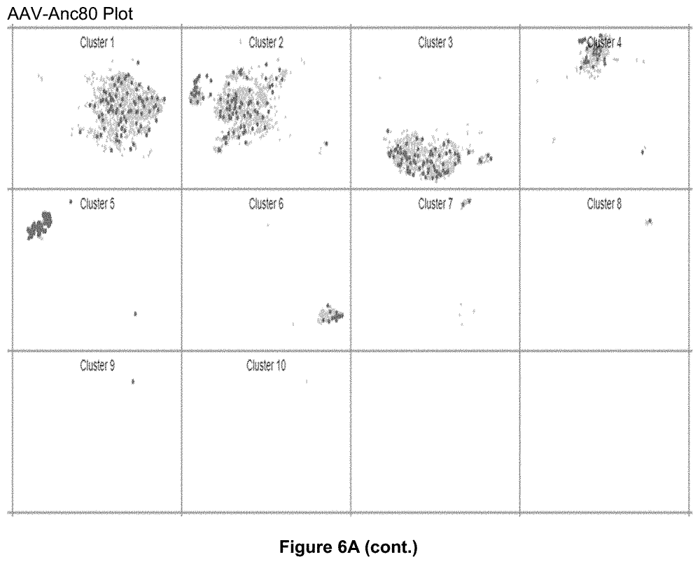

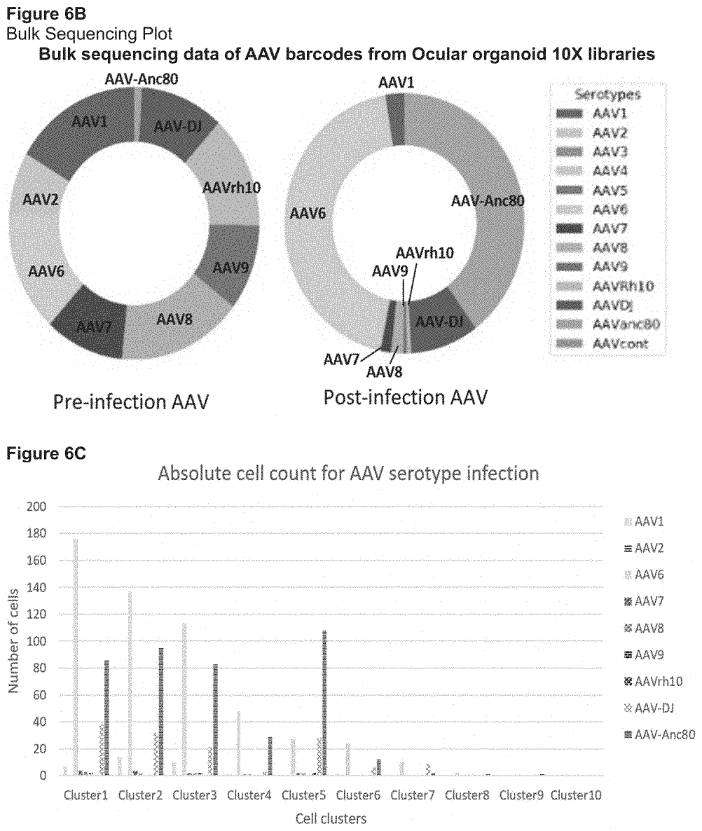

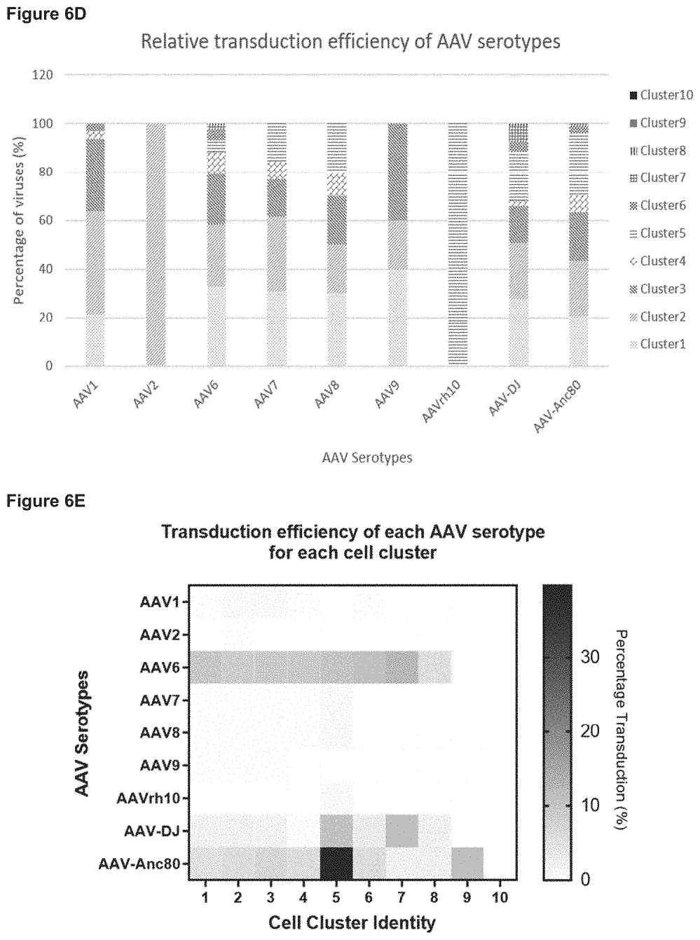

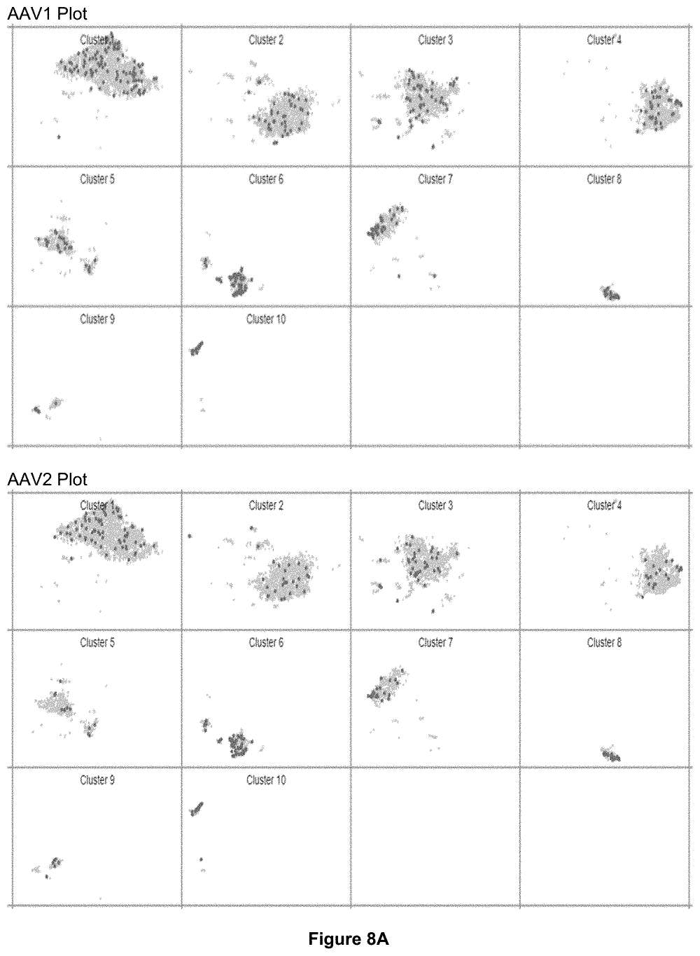

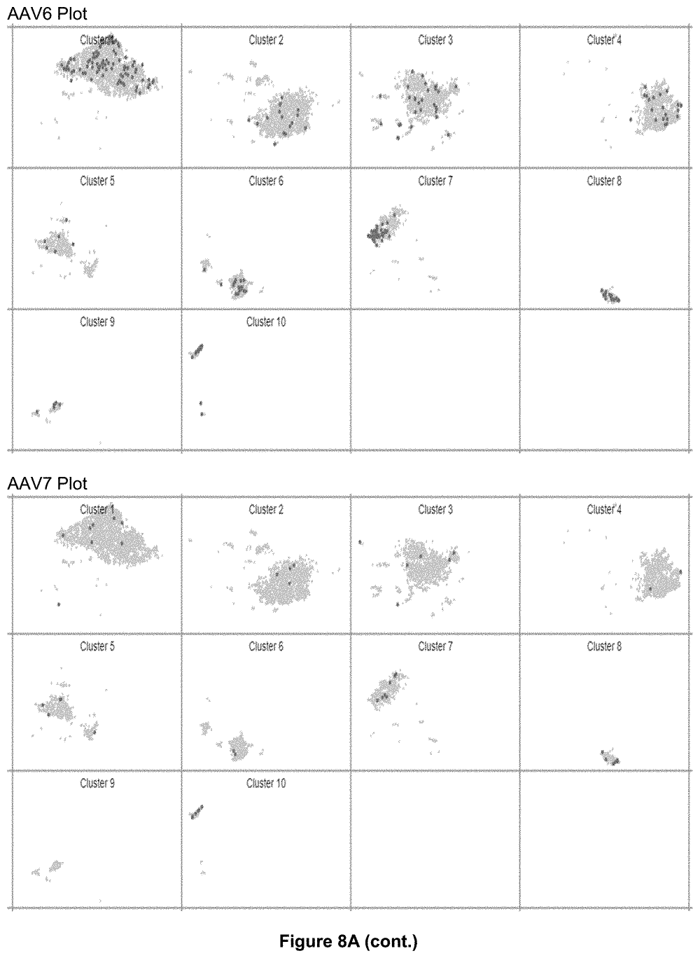

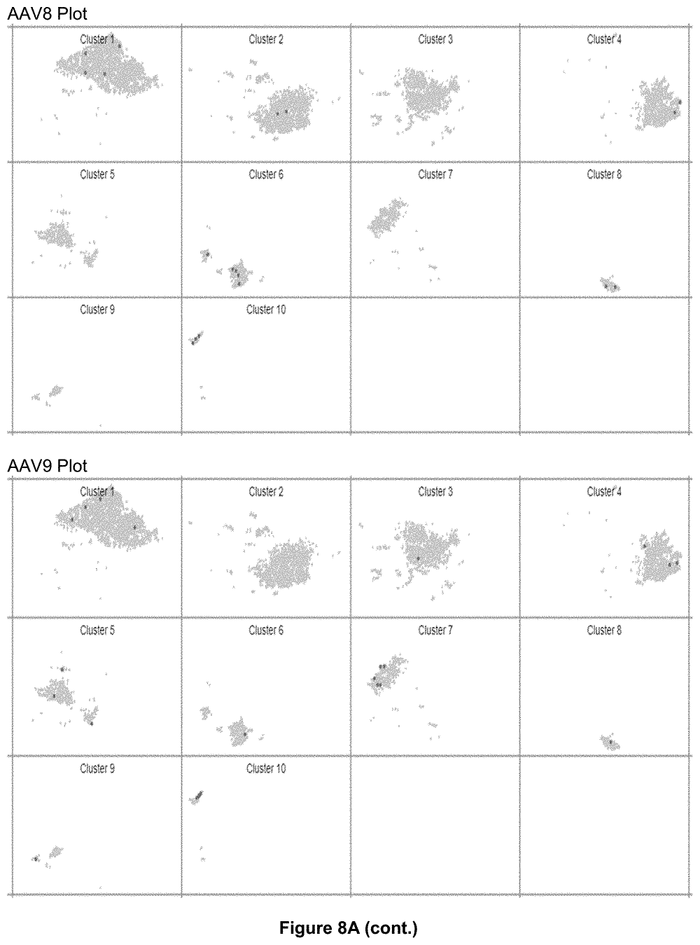

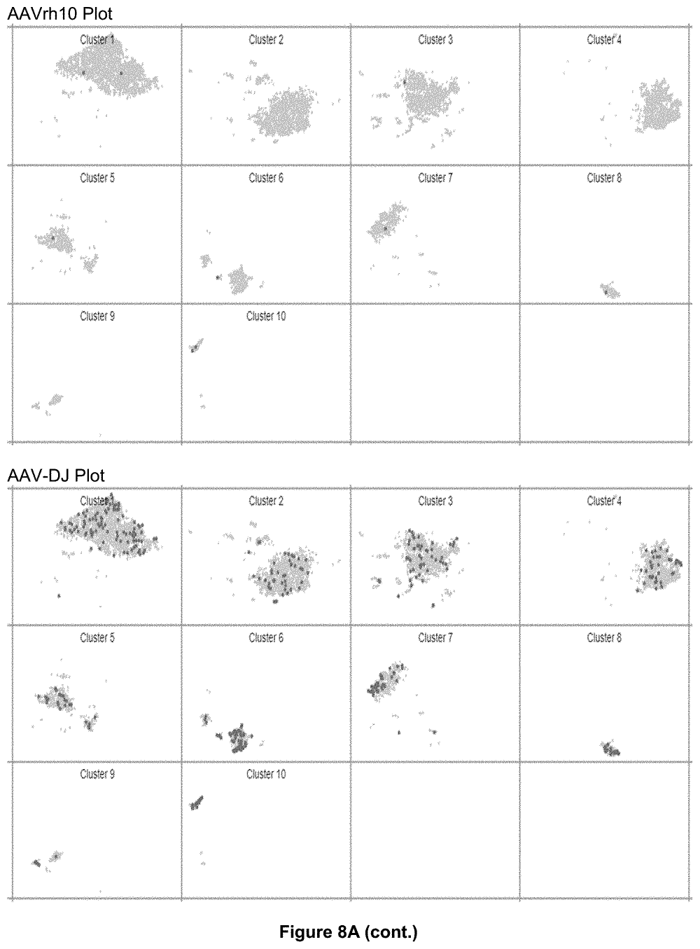

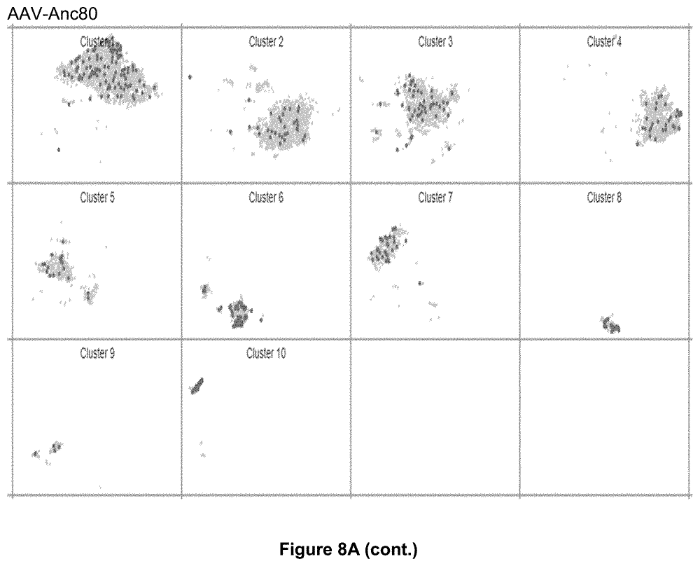

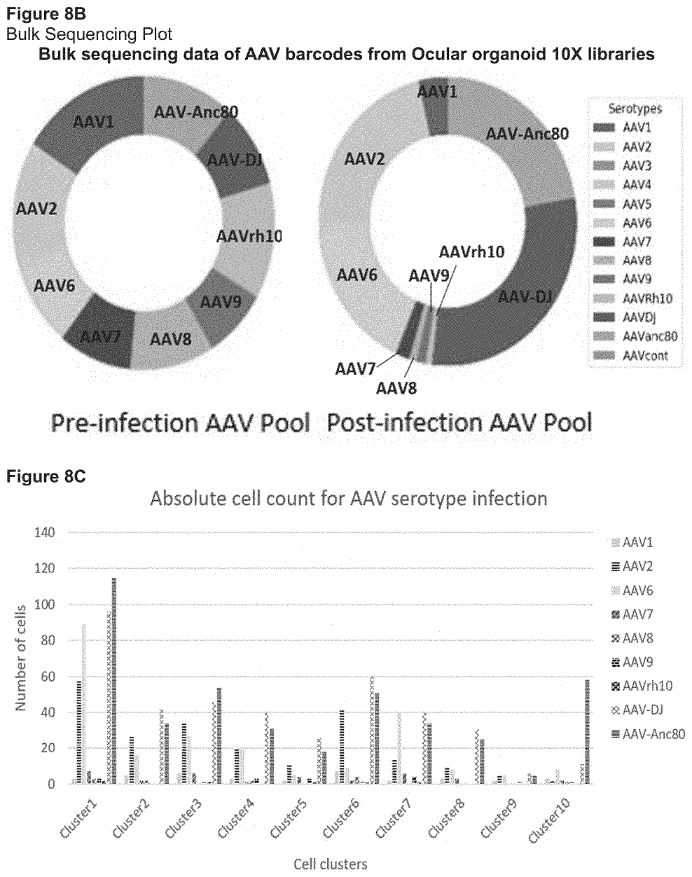

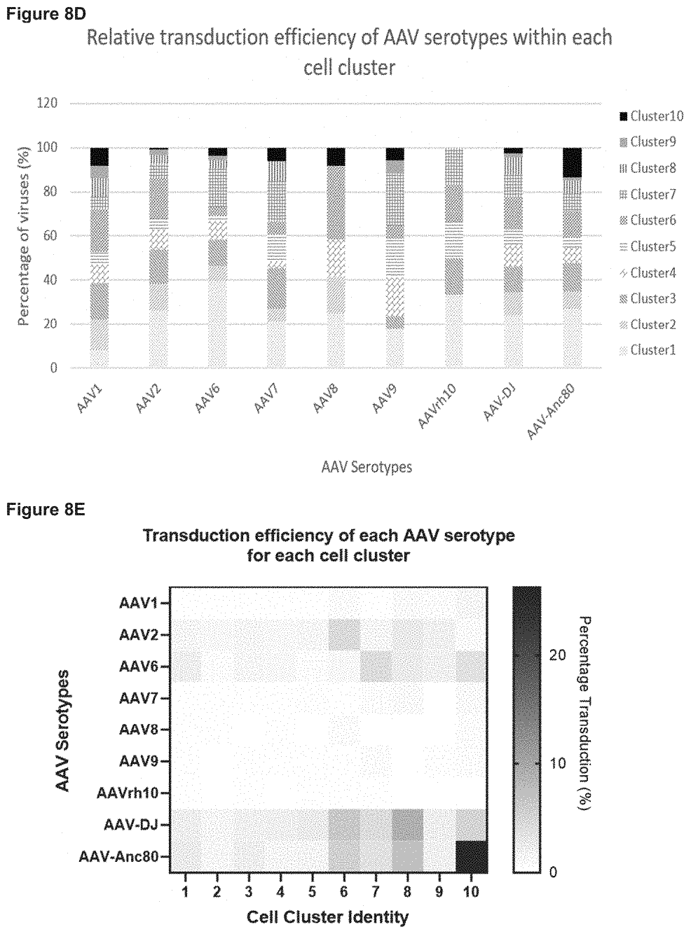

In the Loupe Browser, K-means based clustering were selected to define niche cell population within each type of organoid. The AAV barcoded transcripts can be visualized under Gene/Feature Expression Analysis. The number of cells that are transduced by each serotype in each cell niche are then visualized using the Cell Loupe software and counted, and further tropism analysis (FIGS. 6C-6E and 8C-8E) was conducted in GraphPad Prism.

To determine the transduction efficiency of a specific viral vector against a specific cell niche, the percentage of cells of the specific cell niche which have been detected as positive for the presence of the specific viral vector was calculated.

To calculate the transduction efficiency of a specific viral vector against a specific cell niche, the frequencies with which the presence of the specific viral vector is detected in the cells of the specific cell niche was calculated against the frequencies with which the presence of another viral vector is detected in the cells of the same specific cell niche.

To determine the transduction specificity of a specific viral vector against a specific cell niche relative to other cell niches, the frequencies with which the presence of that specific viral vector is detected in the cells of the specific cell niche was calculated against the frequencies with which the presence of the same specific viral vector is detected in the cells of other specific cell niches.

The t-Stochastic Neighbor Embedding (t-SNE) plot of 5849 cells from human ocular organoids derived from H1 human ES cells are separated into 10 distinct clusters by K-means. The sequenced FASTQ files are processed by a modified Cell Ranger pipeline and visualized on the Cell Loupe software, the mean reads per cell is 122688 and the median genes per cell is 1022.

The t-Stochastic Neighbor Embedding (t-SNE) plot of 15466 cells from human cerebral organoids derived from H1 human embryonic stem cells, separated into 10 distinct clusters by K-means. The sequenced FASTQ files are processed by a modified Cell Ranger pipeline and visualized on the Cell Loupe software, the mean reads per cell is 23315 and median genes per cell is 902.

Experimental Result

Study Design

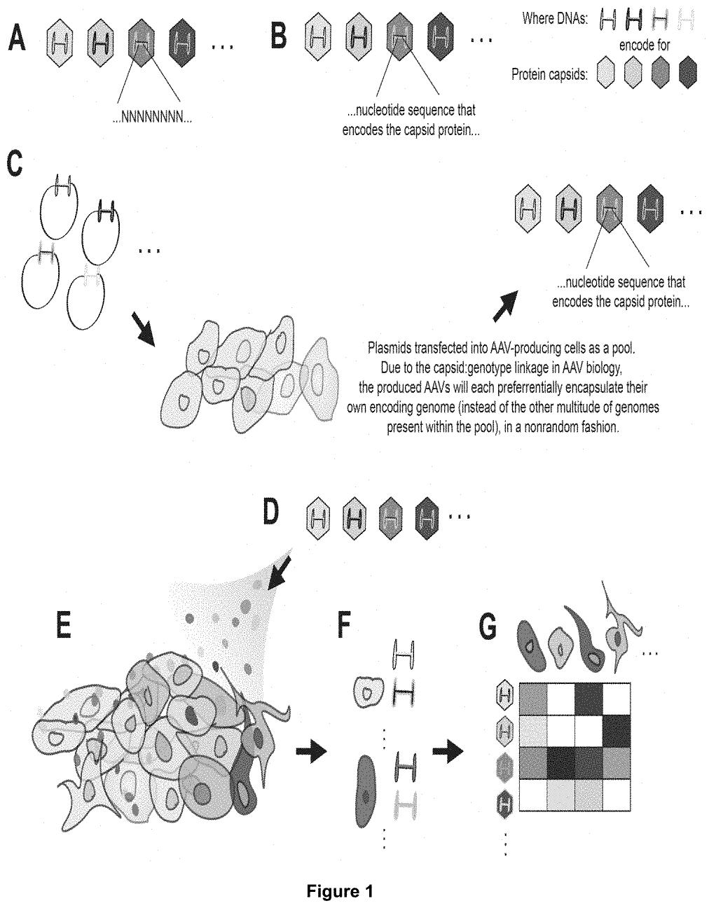

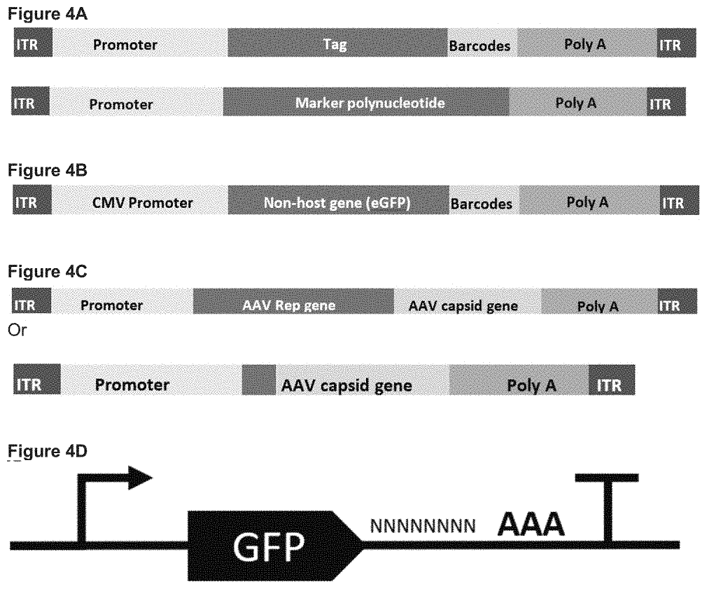

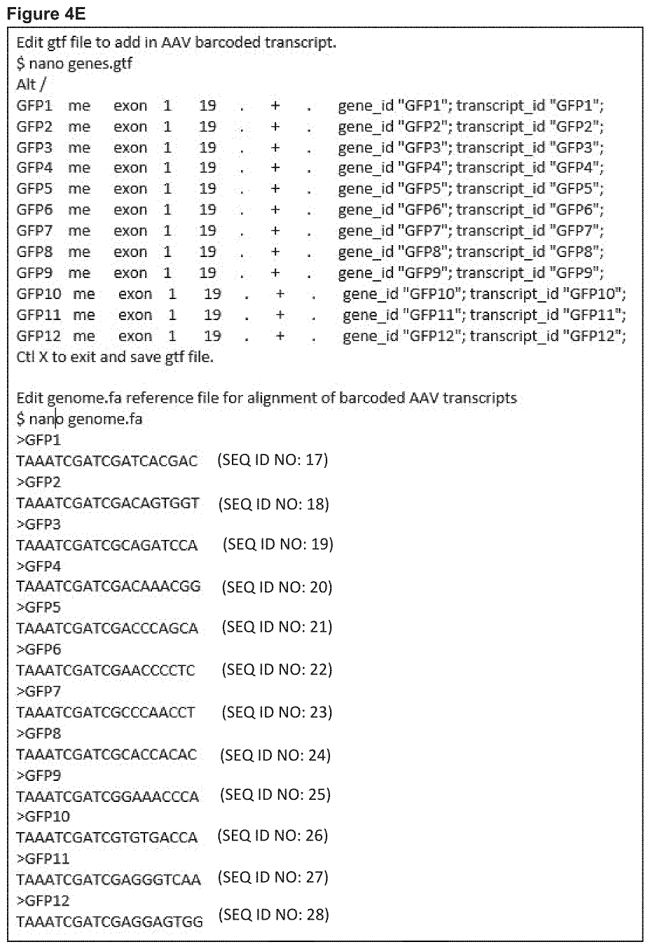

A new framework is provided for assessing multiplex viral tropism in complex tissues in a high-throughput manner and at single-cell resolution. First, panels of AAV serotypes were generated, where the AAV cargo is uniquely differentiable from each other. Specifically, individual packaging vectors of each AAV serotype each contains an eGFP transgene that is barcoded by a unique 8 base pair (bp) sequence at its 3′ end prior to the polyadenylation tail sequence (FIG. 4D). AAVs were produced from these barcoded packaging plasmids, and the pooled AAVs were used to transduce heterogeneous populations of cells within human ocular and cerebral organoids. Following transduction and cargo expression within infected cells, the organoids were dissociated for single-cell sequencing to identify the cell type and the AAV barcodes that infected the particular cell (FIG. 1). Modifications made to the genome reference file and genome transcript file allowed for the AAV barcoded transcripts to be aligned and clustered together with the RNA transcriptomics data for the assignment and visualization of each AAV serotype transcript to individual cells in the Loupe Browser software at single-cell resolution.

Barcoded AAVs Transduce Diverse Tissue Subtypes in Human Ocular and Cerebral Organoids

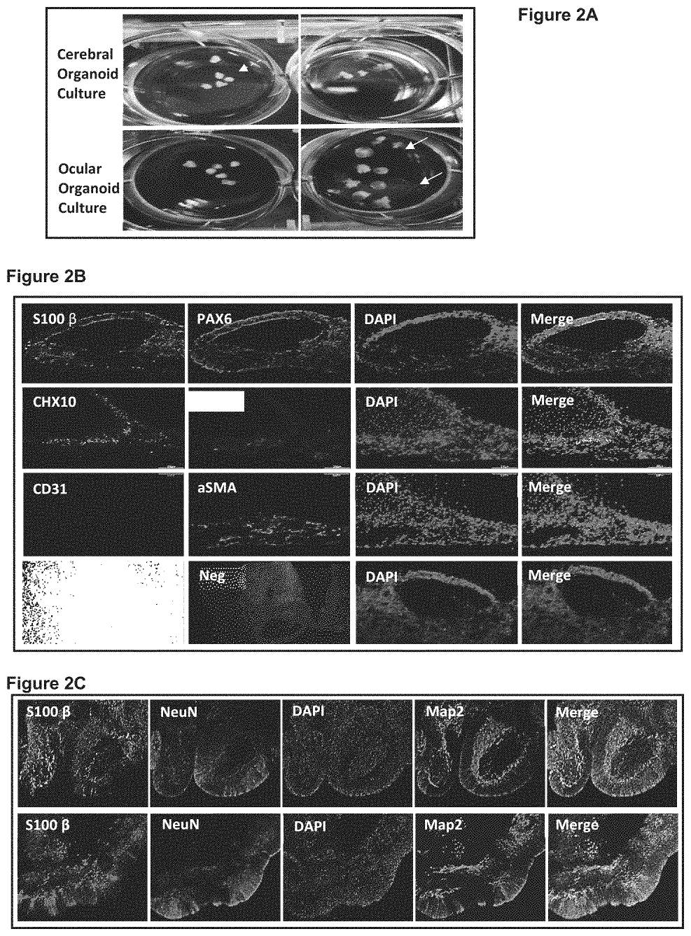

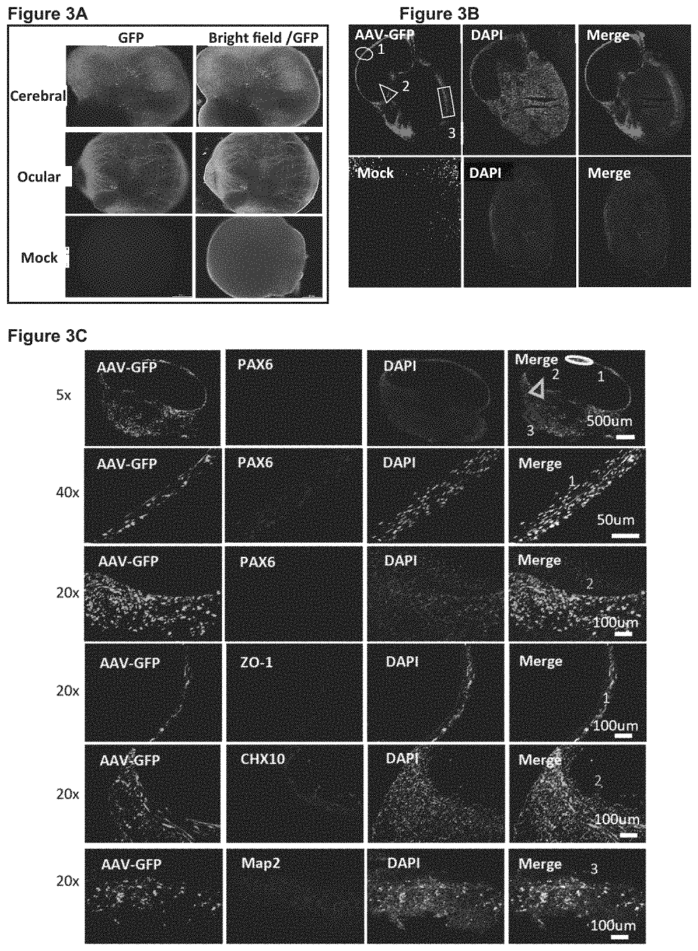

Human ocular and cerebral organoids serve as exemplary models that represent the complexity of human tissues comprising multiple cellular subtypes. The organoids were cultured by differentiating H1 and H9 lineage of human ES cells on petri dishes for 6 weeks (FIG. 2A). The ocular organoids were characterized by immuno-staining for common ocular tissue cellular markers S10013, PAX6, CHX10, RAX, CD31 and aSMA (FIG. 2B), and the cerebral organoids were characterized by immuno-staining with common neural tissue cellular markers S100β, NeuN and Map2 (FIG. 2C). Both the ocular and cerebral organoids express different cellular markers in distinct cellular layers, indicating heterogenous tissue subtypes within the organoids. The barcoded AAV pools (1×10¹⁰ (vector genomes (vg)/per serotype) were then administered upon the cerebral and ocular organoids. Culturing these organoids for a further 7 days resulted in strong GFP-positive signals in cells within most regions of the organoids indicating transduction and expression of the GFP cargo common among the pooled AAVs (FIGS. 3A-3B). Co-localization of eGFP with several different cellular markers also confirmed that the pooled AAVs transduced diverse tissue subtypes within the human ocular and cerebral organoids (FIG. 3C).

Single Cell RNA Transcriptomics Clustering and Assignment of AAV Barcoded mRNA Transcripts in Transduced Ocular and Cerebral Organoids at Single Cell Resolution

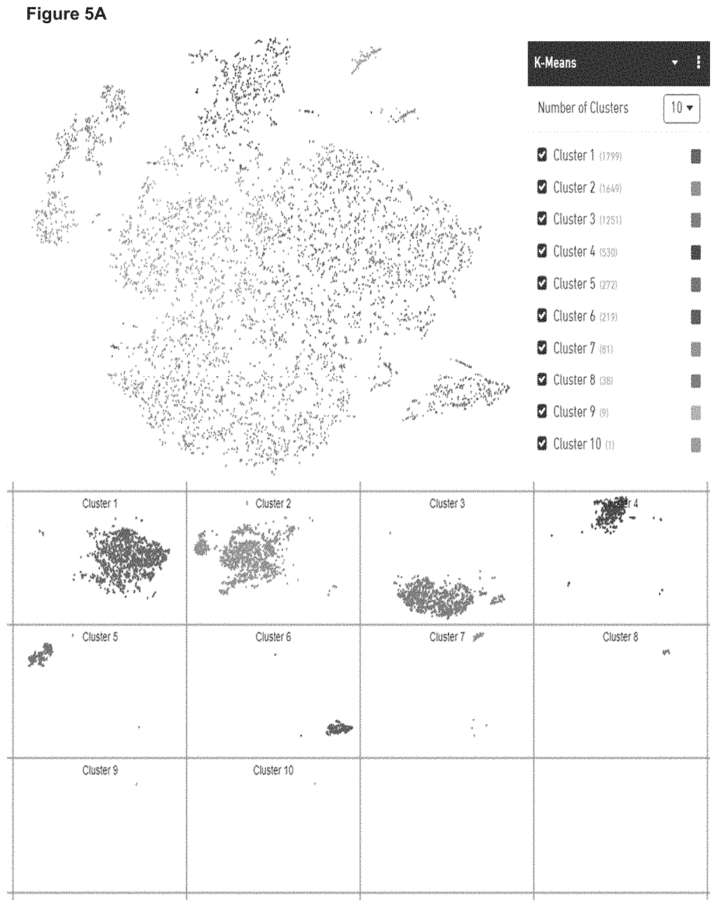

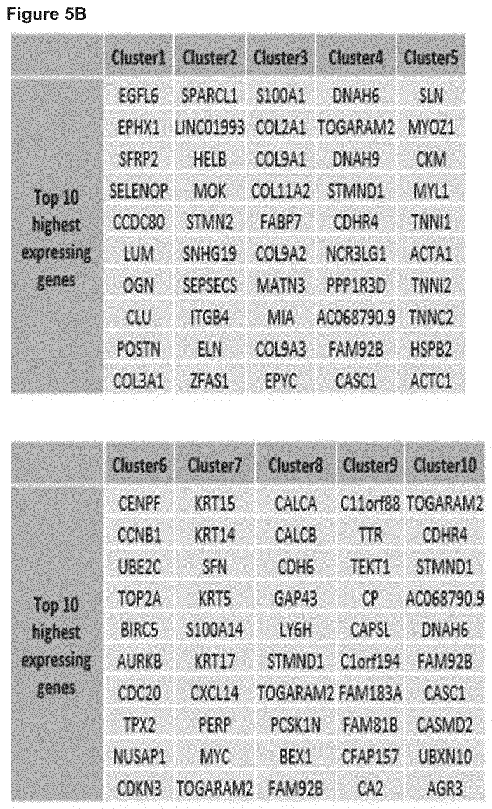

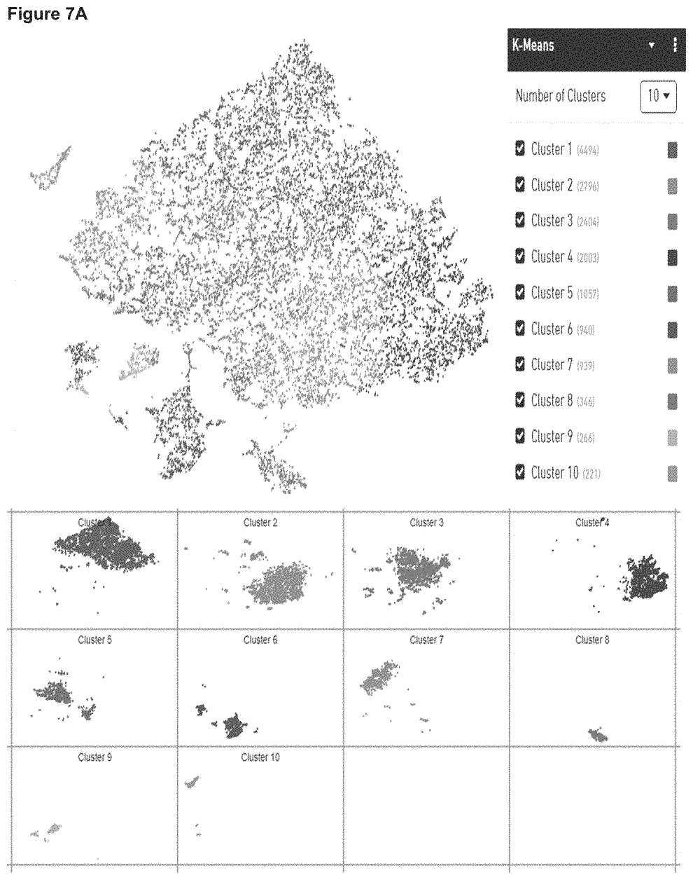

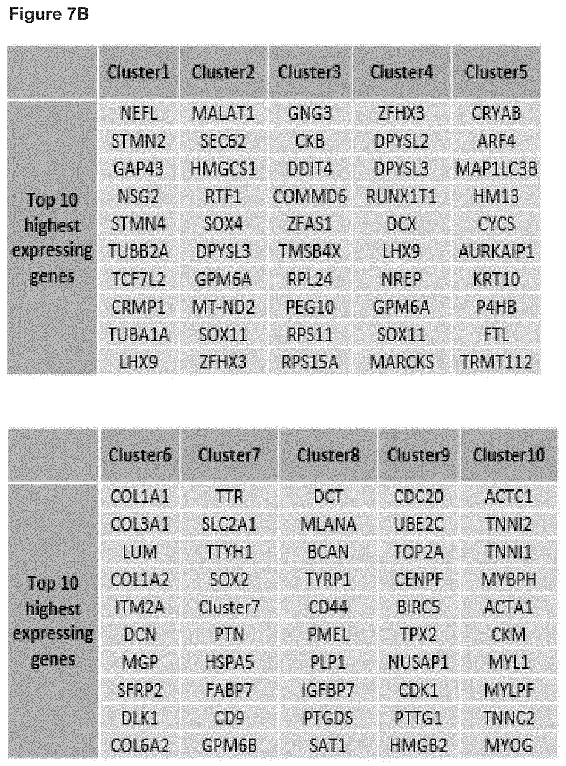

After the human ocular organoids were transduced by the AAV libraries as described above, they were trypsinized into single cells as input for single-cell library preparation and sequencing, see materials and methods. For ocular organoid, the transcriptomes of 5849 cells within the organoid (Sample number tested=3) were profiled with mean reads per cell at 122688 and median genes per cell at 1022. Using K-means clustering, we were able to define 10 clusters of cells within the ocular organoids based on their transcriptomic profile (FIG. 5A and Supplementary Data II). Each cluster of cells were uniquely identified by their top 10 expressing genes within the cluster (FIG. 5B and Supplementary Data II). FIG. 6A shows individual plots of individual serotype for each cluster, the plots representing the assignment of all the AAV transcripts at single cell resolution. To demonstrate that the methodology is easily applied on different complex tissues, the same single-cell tropism assay was also performed on human cerebral organoids, which contain different populations of cell types compared to the ocular organoids. For the cerebral organoid, single-cell sequencing profiled the transcriptomes of 15466 cells within the organoid (Sample number tested=3) with mean reads per cell at 23315 and median genes per cell at 902. Similarly, using K-means clustering, we were able to define 10 clusters of cells within the ocular organoids based on their transcriptomic profile (FIG. 7A and Supplementary Data III). Each cluster of cells were uniquely identified by their top 10 expressing genes (FIG. 7B and Supplementary Data III). FIG. 8A shows individual plots of individual serotype for each cluster, the plots representing the assignment of all the AAV transcripts at single cell resolution.

Next, the multiplex tropism assessment technology to conduct bulk sequencing of the GFP barcodes was compared. This is a method that is used to examine AAV transduction in bulk tissues. Data from bulk sequencing of ocular organoids is in concordance with the single-cell sequencing data aggregated across cells (Table 3 and FIG. 6B), with AAV-Anc80, AAV6 and AAV-DJ being the top 3 AAV serotypes that most efficiently transduce the ocular organoids in bulk or in aggregate among single-cells. Similarly, the data from bulk sequencing of the cerebral organoids also aligns with the single cell sequencing data with AAV2, AAV6, AAV-DJ and AAV-Anc80 as the top 4 AAV serotypes that can most efficiently transduce the cerebral organoids (Table 4 and FIG. 8B).

TABLE 3
Data from bulk sequencing of ocular organoids

| | Bulk | Single cell |
|---|---|---|
| | sequencing counts | sequencing counts |
| | for ocular | for ocular |
| AAV Barcodes | organoid (%) | organoid (%) |
| A701 (AAV1) | 2.55 | 2.85 |
| A702 (AAV2) | 0.19 | 0.09 |
| A706 (AAV6) | 44.02 | 46.45 |
| A707 (AAV7) | 1.40 | 1.12 |
| A708 (AAV8) | 1.65 | 0.87 |
| A709 (AAV9) | 0.49 | 0.43 |
| A710 (AAVrh10) | 0.58 | 0.17 |
| A711 (AAVDJ) | 9.21 | 11.93 |
| A712 (AAVAnc80) | 39.88 | 36.07 |

TABLE 4
Data from bulk sequencing of cerebral organoids

| | Bulk | Single cell |
|---|---|---|
| | sequencing counts | sequencing counts |
| | for cerebral | for cerebral |
| AAV Barcodes | organoid (%) | organoid (%) |
| A701 (AAV1) | 3.63 | 2.62 |
| A702 (AAV2) | 21.72 | 16.08 |
| A706 (AAV6) | 17.88 | 16.45 |
| A707 (AAV7) | 1.76 | 2.40 |
| A708 (AAV8) | 0.91 | 0.87 |
| A709 (AAV9) | 1.17 | 1.24 |
| A710 (AAVrh10) | 0.80 | 0.44 |
| A711 (AAVDJ) | 29.60 | 28.97 |
| A712 (AAVAnc80) | 22.33 | 30.93 |

Importantly, by extracting the read counts of the different AAV serotypes transcripts in each cell cluster, it is possible to visualize the absolute (FIGS. 6C and 8C) and relative (FIGS. 6D and 8D) transduction efficiency of each AAV serotype across heterogeneous cell types within the organoids. For ocular organoids, when normalized against GAPDH across all clusters, AAV-Anc80 is identified as the most efficient serotype for targeting cell clusters 5 representing retinal-like cell types (RDH5hi, MITFhi), while AAV6 and AAVDJ are the most efficient serotype transducing cell cluster 7 representing epithelium-like cell types (TP63hi, KRT5hi) and cluster 8 representing neural stem-like cell types (PAX6hi, SOX2hi, MAP2hi) (FIG. 6E). Similarly, for the cerebral organoid, when normalized against GAPDH across all cell clusters, AAV2, 6 and Anc-80 were identified as serotypes that can efficiently transduce cluster 6 representing brain meningeal-like cells (DCNhi, SOX2hi, PAX2hi) while AAV6 and AAVDJ most efficiently transduce cluster 7 representing midbrain dopaminergic-like cells (RSPO2hi, SOX2hi, PAX6hi). In addition, the result suggests that AAVDJ is the most efficient serotype for cluster 8 representing astroglia or Schwann-like cell types (S100Bhi) and AAV-Anc-80 is the most efficient serotype for cluster 10 representing microglial-like cells (UCP2hi) (FIG. 8E). These results show that the single-cell AAV tropism assay identifies different AAV serotypes with preferential tropism towards each subset of human cell types within the ocular or cerebral organoids.

To date, most of the published AAV tropism assays utilize low-resolution methods to perform relative comparison between a few AAV serotypes, conducted either in vitro using homogenous cell lines or in vivo using hulk tissue organs. The present disclosure presents a pipeline that enables high-throughput multiplexing of AAV libraries for relative comparison of transduction efficacies at single-cell resolution. Such a pipeline also allows the assessment of AAV tropism for single cell niches in a high throughput manner which is increasingly important as single cells studies identified more focused cell niches that contribute to disease pathologies.

Tropism of a library of AAV serotypes consisting of natural (AAV1, 2, 6, 7, 8, 9, and rh10) and engineered AAVs (DJ and Anc-80) is evaluated for their transduction efficacy, across different single-cell niches within the same tissue organoid simultaneously. High-resolution quantification of every AAV serotype mRNA transcripts that are present in each single cell reveals the AAV serotype(s) that has preferential tropism towards individual cell types. Although the current demonstrated data employs the use of only 9 serotype variants, the assay could likely support substantially more variants as the barcoding strategy allows for simple scaling up the current 8-nt barcoding can support 65K unique barcodes and serotypes, before implementation of error-tolerating or error-correcting encoding). The method can also be applied beyond ocular or cerebral organoids to any tissue in vitro or in vivo, especially when the targeted cellular subtypes have established cell type markers to facilitate annotation. This method can potentially be employed for clinical development by refining the selection of AAV serotypes for precise gene delivery to diseased tissues.

The present disclosure presents a technology pipeline that enables multiplex measurement of AAV transduction efficiency and specificity for each cell type within a heterogeneous population is developed. AAV serotypes are barcoded according to a new design principle as disclosed herein, and the AAV library is applied on complex mixtures of cell types. Single-cell sequencing is conducted to identify both the cell type and the AAV barcodes each single cell contains. The data obtained from sequencing is deconvoluted into matrices of AAV serotype versus human cell types. Human organoids were selected for testing the technology pipeline as they recapitulate certain structural and cellular complexity of, for example, the human brain and eye. The technology pipeline identified the efficiency and specificity that each AAV serotype transduces individual cell types found within the organoids. This technology pipeline also enables a more comprehensive interrogation of delivery vector biodistribution that will impact safety and efficacy profiles of the therapeutic product.

1. A method for assessing the transduction efficiency and/or specificity of vectors at single cell level, said method comprising:
a) providing a plurality of different vectors,
b) transducing a heterogeneous population of cells with the plurality of different vectors;
c) partitioning the heterogeneous population of cells into a plurality of compartments, wherein each compartment comprises a single cell from the heterogeneous population of cells;
d) subjecting each partitioned cell to nucleotide sequencing; and
e) detecting the presence of the any one or more of the different vectors in each partitioned cell.

2. The method of claim 1, wherein the method further comprises:
(f) classifying each partitioned cell into a specific cell type based on gene expression patterns and/or epigenetic features of said cell, as determined using sequencing results obtained in step d).

3. The method of claim 1, wherein the transduction efficiency of a specific vector against a specific cell type is determined by a percentage of cells of the specific cell type which have been detected positive for the presence of the specific vector; and/or wherein the transduction efficiency of a specific vector against a specific cell type is assessed by comparing frequencies with which the presence of said specific vector is detected in the cells of said specific cell type, against frequencies with which the presence of another vector is detected in the cells of said specific cell type; and/or wherein the transduction efficiency of a specific vector against a specific cell type is assessed by comparing the frequencies with which the presence of said specific vector is detected in the cells of said specific cell type, against the frequencies with which the presence of another vector is detected in the cells of said specific cell type.

4.-5. (canceled)

6. The method of claim 1, wherein each of the plurality of different vectors comprises an oligonucleotide barcode sequence, wherein the barcode sequence is different between any two different vectors; and/or wherein the barcode sequence is located on an expression cassette in the vector, wherein the expression of the cassette results in the production of an RNA molecule comprising the barcode sequence, wherein the RNA molecule further comprises a polyadenylation tail.

7. (canceled)

8. The method of claim 6, wherein the barcode sequence is located on a region of the RNA molecule which allows the barcode sequence to be sequenced.

9. The method of claim 8, wherein the barcode sequence is within a distance of 98 nucleotides from the polyadenylation tail.

10. The method of claim 6, wherein the barcode sequence is 5, 6, 7, 8, 9, 10, 11, 12, 13, 14, 15, 16, 17, 18, 19, 20, 21, 22, 23, 24, 25, 26, 27, 28, 29, 30 nucleotides in length; and/or wherein the barcode sequence is 8 nucleotides in length.

11. (canceled)

12. The method of claim 1, wherein each of the plurality of different vectors comprises a marker polynucleotide, wherein the marker polynucleotide is different between any two different vectors; and wherein the marker polynucleotide encodes for one or more proteins, said one or more proteins when expressed form a protein envelope which encapsulate the marker polynucleotide, so that after transfection of the vector, each marker polynucleotide is encapsulated by the one or more proteins which the marker polynucleotide encodes for; and/or wherein the marker polynucleotide is located on an expression cassette in the vector, wherein the expression of the cassette results in the production of an RNA molecule comprising the marker polynucleotide, wherein the RNA molecule further comprises a polyadenylation tail; and/or wherein the marker polynucleotide is a viral-capsid-encoding gene, wherein the capsid expressed by the marker polynucleotide encapsulates the marker polynucleotide.

13.-14. (canceled)

15. The method of claim 12, wherein the viral-capsid-encoding gene is specifically an AAV-capsid-encoding gene.

16. The method of claim 1, wherein step e) comprises detecting the presence of one or more marker sequences specific to each different vector; wherein when each vector comprises a unique barcode sequence, said one or more marker sequences comprise the barcode sequence; wherein when each vector comprises a unique marker polynucleotide, said one or more marker sequences comprise the marker polynucleotide.

17. The method of claim 16, wherein step e) comprises matching the sequence reads obtained in step d) with a reference data set.

18. The method of claim 17, wherein the reference data set comprises the genomes and/or the transcriptomes of the plurality of different viral vectors, and/or the barcodes comprised in the plurality of different viral vectors, and/or the marker polynucleotides comprised in the plurality of different viral vectors.

19. The method of claim 1, wherein the compartments are oil droplets.

20. The method of claim 1, wherein the nucleotide sequencing is RNA sequencing and/or DNA sequencing.

21. (canceled)

22. The method of claim 1, wherein the vectors are selected from the group consisting of: a viral vector, a pseudo-virus vector, a virus-like particle vector, a liposome vector, an exosome vector, a nanoparticle, and combinations thereof; wherein the vectors comprise DNA, RNA, modified RNA, modified DNA, or combinations thereof; and/or wherein the vectors comprise viral vectors, wherein the viral vectors are selected from the group consisting of: an adenoviral vector, an Adeno-associated virus (AAV) vector, a lentiviral vector, a coronavirus vector, an enterovirus vector, a retroviral vector, or a combination thereof.

23. (canceled)

24. The method of claim 22, wherein the viral vectors are AAV vectors and/or wherein the viral vectors are selected from the group consisting of: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV9), AAV type 10 (AAV10), AAV type 11 (AAV11), AAV type 12 (AAV12), AAV type 13 (AAV13), rh10, AAVDJ, AAVAnc80, AAV-PHP.S, AAV-PHP.eB, AAV-LK03, AAV2-7m8, AAV variants thereof, and combinations thereof.

25. (canceled)

26. The method of claim 22, wherein the plurality of different viral vectors comprises viral vectors of different families, viral vectors of different genera, viral vectors of different species, viral vectors of different serotypes, viral vectors thereof carrying different mutations, or combinations thereof.

27. The method of claim 1, wherein the heterogeneous population of cells comprise plant cells, animal cells, fungal cells, or combinations thereof.

28. The method of claim 27, wherein the heterogeneous population of cells comprise mammalian cells; and/or the heterogeneous population of cells comprise human cells; and/or the heterogeneous population of cells are comprised in an animal or human subject when being transduced.

29.-32. (canceled)

33. The method of claim 27, wherein the one or more cultured organoids are selected from the group consisting of ocular organoid, cerebral organoid, epithelial organoid, kidney organoid, lung organoid, pancreas organoid, cardiac organoid, and hepatic organoid.
